(19) United States
(12) Patent Application Publication
(10) Pub. No.: US 2020/0354327 A1
(43) Pub. Date: Nov. 12, 2020

(54) COMPOUND HAVING STAT3 INHIBITORY ACTIVITY AND USE THEREOF

(71) Applicant: CYTUS H&B CO., LTD., Cheongju-si Chungcheongbuk-do (KR)

(72) Inventors: Chung Gi LEE, Yongin-si, Gyeonggi-do (KR); Sang Kyu YE, Seoul (KR); Byung Hak KIM, Seoul (KR)

(21) Appl. No.: 16/760,436

(22) PCT Filed: Oct. 31, 2017
(86) PCT No.: PCT/KR2017/012151

§ 371 Date: Jul. 2, 2020

(51) Int. Cl. C07D 271/07; C07D 239/88; A61P 35/04; A61P 17/02; C07D 213/53

(52) U.S. Cl. CPC C07D 271/07; C07D 239/88; C07D 213/53; A61P 17/02; A61P 35/04

(57) ABSTRACT

The present invention relates to a compound exhibiting STAT3 inhibitory activity, or a pharmaceutically acceptable salt, solvate or hydrate of the same, and pharmaceutical uses of these. The compounds of the present invention efficiently inhibit the abnormal activity of STAT3 associated with various diseases and thus can be usefully utilized for the prevention and treatment of various STAT3-related diseases associated with cancer, autoimmune diseases, inflammatory diseases and the like.

### TECHNICAL FIELD

The present invention relates to a compound exhibiting STAT3 inhibitory activity, or a pharmaceutically acceptable salt, solvate or hydrate of the same, and pharmaceutical uses of these.

### BACKGROUND ART

In previous studies, the present inventors have confirmed that signal transducer and activator of transcription 3 (STAT3) plays an important role in the stability of the hypoxia inducible factor-1alpha (HIF-1alpha) protein and interacts with HIF-1alpha of human kidney cancer cells to improve HIF-1-mediated expression of vascular endothelial growth factor (VEGF) (Jung JE, et al. FASEB J. 19, 1296-1298 (2005). In addition, the present inventors have reported that caffeic acid derivatives effectively inhibit the expression of VEGF gene by inhibiting the activity of tyrosine-705 of STAT3 (Jung J E, et al. Carcinogenesis. 28, 1780-1787 (2007)). Moreover, the present inventors have reported that the transcriptional activity of cyclin D1, that is known to regulate cell division and cell proliferation, is regulated dependent on the activity of tyrosine-705 of STAT3 (Won C, et al. Anticancer Res. 30, 481 -488 (2010)). These facts demonstrate that STAT3 regulates hypoxia-mediated VEGF expression in solid cancer cells to be an important up-regulator of angiogenesis essential for the proliferation of cancer cells and regulates the cell cycle of cancer cells to be involved in the division and proliferation of cancer cells.

STATs are transcription factors which are activated by being phosphorylated with januse kinase (JAK), epidermal growth factor receptor (EGFR), and platelet-derived growth factor receptor (PDGFR), which are a kind of receptor tyrosine kinase (Darnell J E Jr. Science. 277, 1630-1635 (1997)). STATs activated by phosphorylation are known to form dimers, migrate into the nucleus, bind near the promoter of the target gene, and thus induce transcription of various genes associated with diseases (Darnell J E Jr. Science. 277, 1630-1635 (1997); Bromberg J F, et al. Cell. 98, 295 -303 (1999)). STAT3, that is a class of STATs protein, is known to be over-activated in various kinds of human tumors including blood cancer and solid cancers (Bromberg J F, et al. Cell. 98, 295-303 (1999)). Overactivated STAT3 is known to promote cancer cellization of mutated cells by promoting the expression of target genes such as Bcl-XL, c-myc, and cyclin D1, that are associated with cancer cell survival, proliferation and growth (Vera J, et al. Prog Biophys Mol Biol. 106, 426-434 (2011); Turkson J. Expert Opin Ther Targets. 8, 409-422 (2004)). In addition, recent reports suggest the possibility that STAT3 inhibitors are potential anti-cancer agents (Duan H, et al. Oncogene. 27, 6720-6728 (2008); Bai L, et al. Int J Cancer. 130, 2693-2702 (2012); Kan C E, et al. Cancer Res. 71, 6930-6939 (2011)).

Throughout this specification, a number of papers and patent documents are referenced and their citations are indicated. The disclosures of cited papers and patent documents are incorporated by reference into the present specification as a whole, and the level of the technical field to which the present invention pertains and the content of the present invention are more clearly described.

### SUMMARY OF INVENTION

### Technical Problem

The present inventors have made extensive efforts to develop novel compounds exhibiting selective STAT3 inhibitory activity for the prevention and treatment of diseases associated with excessive expression or activation of STAT3, such as cancer, autoimmune diseases, and inflammatory diseases. As a result, the present inventors have confirmed that derivatives having 3-phenoxymethyl-1,2,4-oxadiazole or 3-phenoxymethyl-1,2,4-thiadiazole as a skeletal structure are effective in STAT3-related diseases since the derivatives inhibit activation of STAT3, that is, inhibit the phosphorylation thereof to activate caspase-3 and PARP, inhibit the activity of MMPs, and thus effectively inhibit the expression of Twist gene, and completed the present invention.

Accordingly, an object of the present invention is to provide compounds represented by Chemical Formulas 1 to 60, or pharmaceutically acceptable salts, solvates or hydrates thereof.

Another object of the present invention is to provide a pharmaceutical composition for the prevention or treatment of STAT3-related diseases.

Another object of the present invention is to provide a food composition for the prevention or improvement of STAT3-related diseases.

Other objects and advantages of the present invention will become more apparent from the following detailed description, claims and drawings of the invention.

### Solution to Problem

According to an aspect of the present invention, the present invention provides a compound selected from the group consisting of compounds represented by the following Chemical Formulas 1 to 60, or a pharmaceutically acceptable salt, solvate or hydrate thereof:

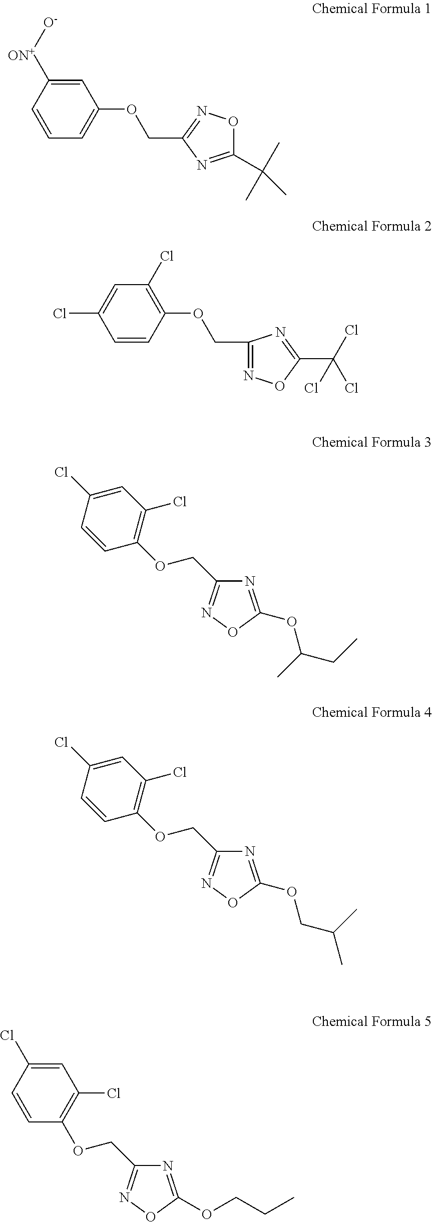

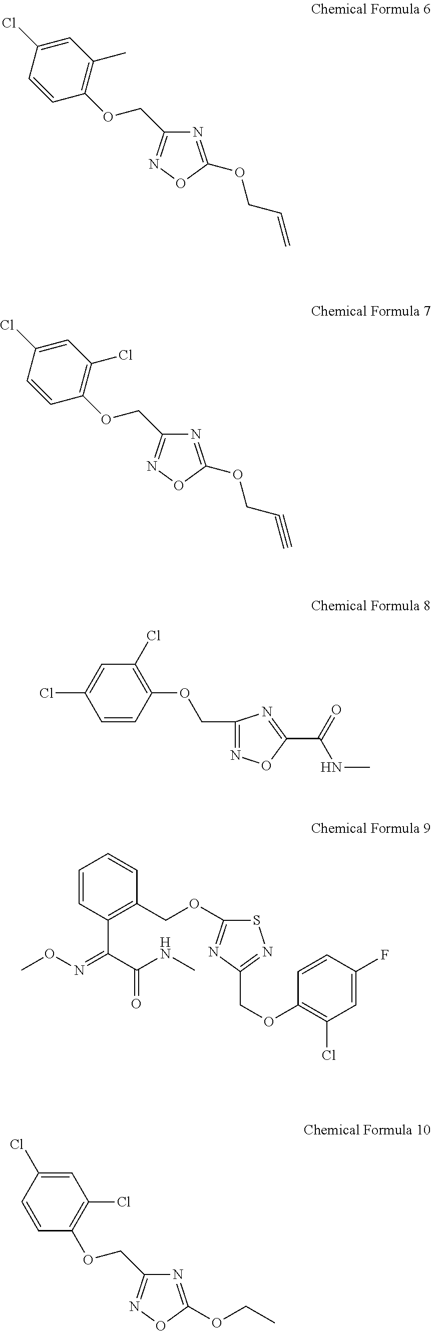

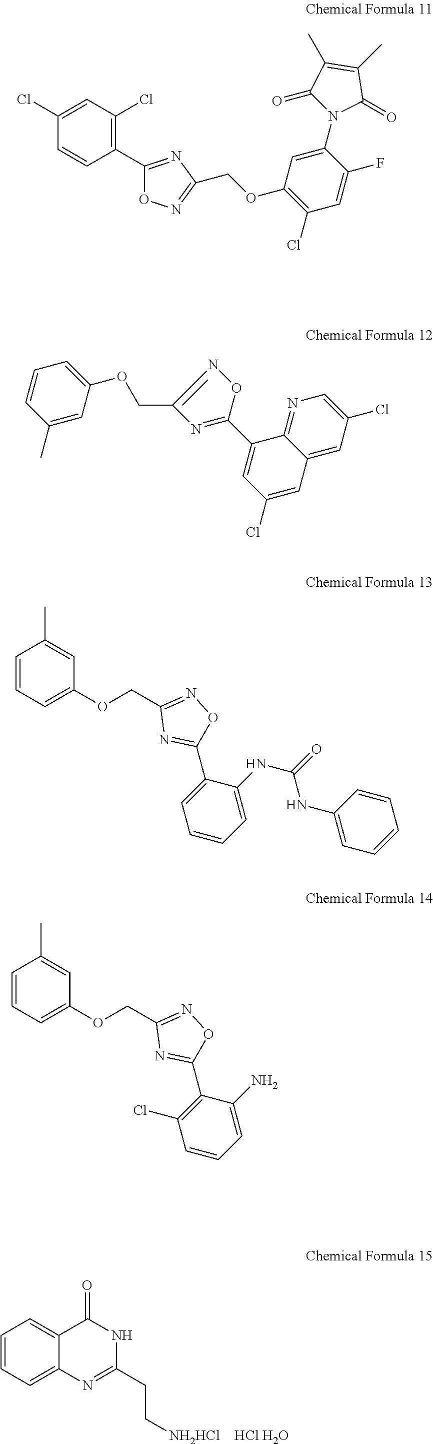

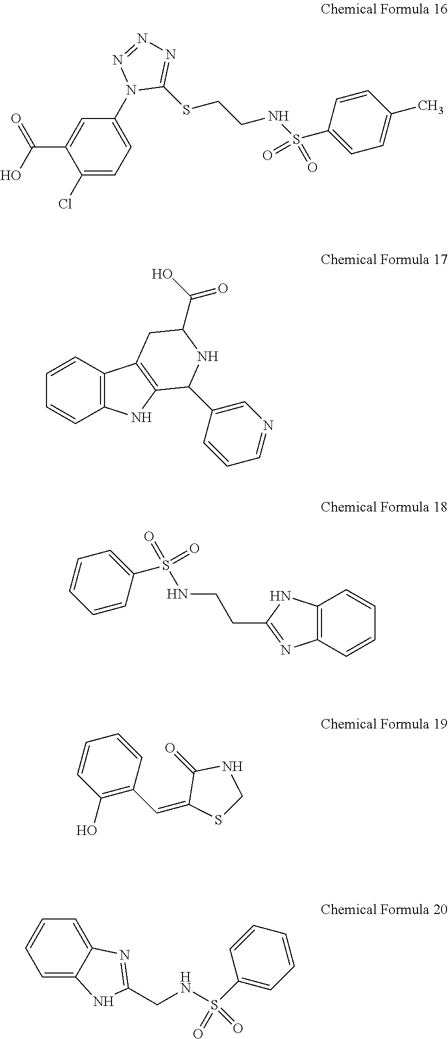 [SMILES: Cc1ccc(S(=O)(=O)NCCSc2nnnn2-c2ccc(Cl)c(C(=O)O)c2)cc1 ; O=C(O)C1Cc2c([nH]c3ccccc23)C(c2cccnc2)N1 ; O=S(=O)(NCCc1nc2ccccc2[nH]1)c1ccccc1 ; O=C1NCS/C1=C/c1ccccc1O ; O=S(=O)(NCc1nc2ccccc2[nH]1)c1ccccc1]

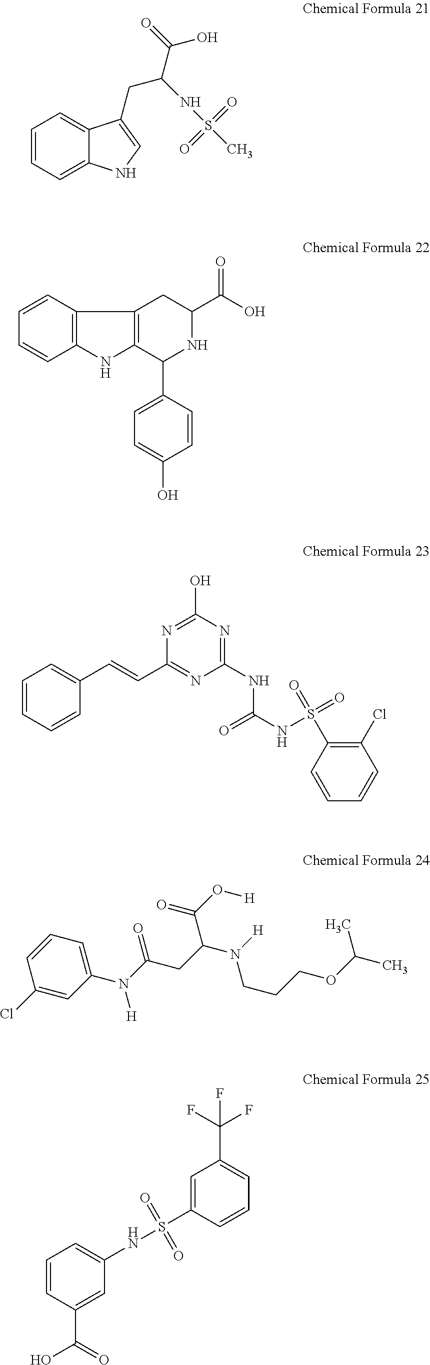 [SMILES: CS(=O)(=O)NC(Cc1c[nH]c2ccccc12)C(=O)O ; O=C(O)C1Cc2c([nH]c3ccccc23)C(c2ccc(O)cc2)N1 ; O=C(Nc1nc(O)nc(/C=C/c2ccccc2)n1)NS(=O)(=O)c1ccccc1Cl ; CC(C)OCCCNC(CC(=O)Nc1cccc(Cl)c1)C(=O)O ; O=C(O)c1cccc(NS(=O)(=O)c2cccc(C(F)(F)F)c2)c1]

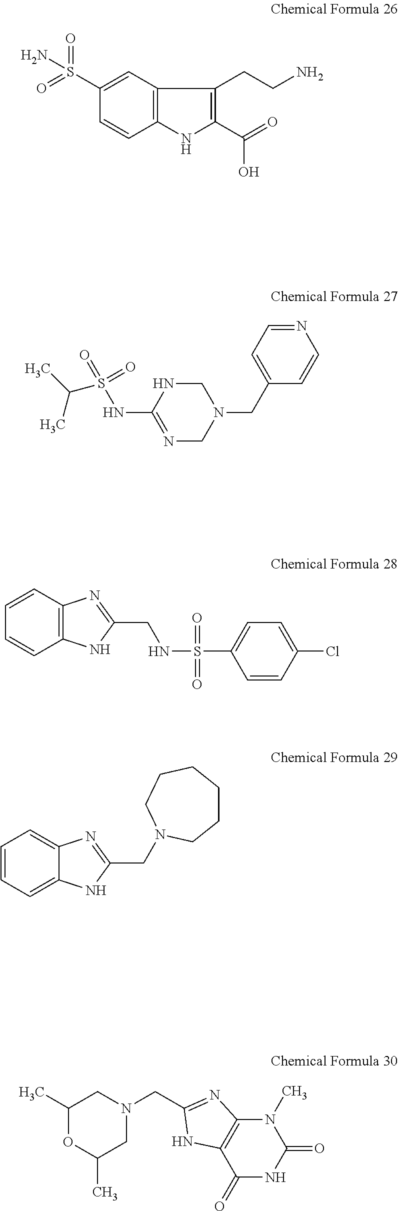 [SMILES: NCCc1c(C(=O)O)[nH]c2ccc(S(N)(=O)=O)cc12 ; CC(C)S(=O)(=O)NC1=NCN(Cc2ccncc2)CN1 ; O=S(=O)(NCc1nc2ccccc2[nH]1)c1ccc(Cl)cc1 ; c1ccc2[nH]c(CN3CCCCCC3)nc2c1 ; CC1CN(Cc2nc3c([nH]2)c(=O)[nH]c(=O)n3C)CC(C)O1]

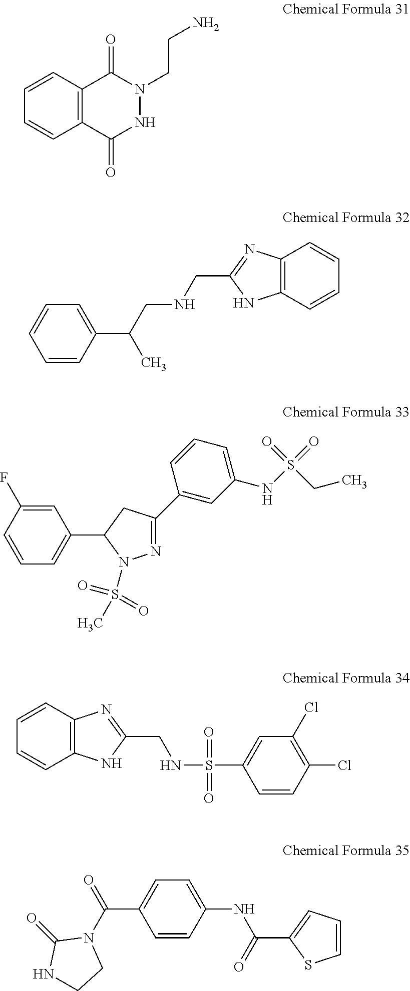 [SMILES: NCCn1[nH]c(=O)c2ccccc2c1=O ; CC(CNCc1nc2ccccc2[nH]1)c1ccccc1 ; CCS(=O)(=O)Nc1cccc(C2=NN(S(C)(=O)=O)C(c3cccc(F)c3)C2)c1 ; O=S(=O)(NCc1nc2ccccc2[nH]1)c1ccc(Cl)c(Cl)c1 ; O=C(Nc1ccc(C(=O)N2CCNC2=O)cc1)c1cccs1]

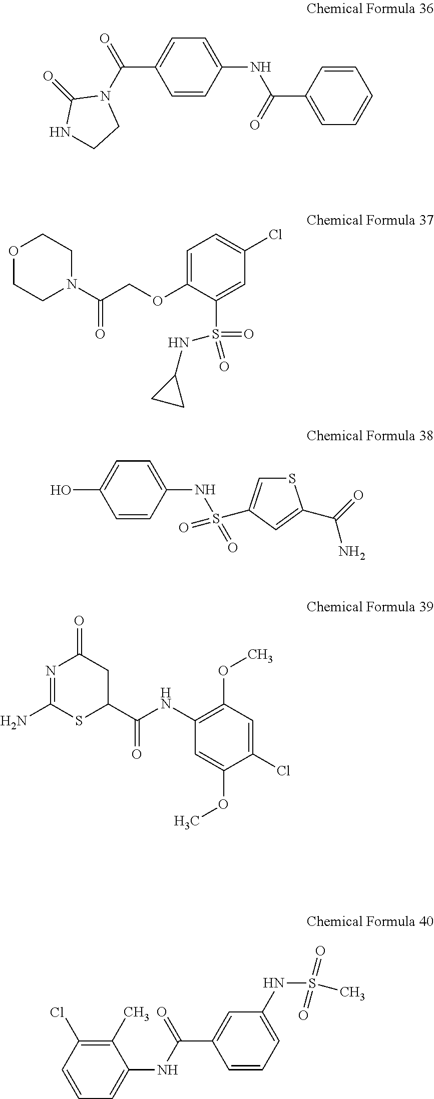 [SMILES: O=C(Nc1ccc(C(=O)N2CCNC2=O)cc1)c1ccccc1 ; O=C(COc1ccc(Cl)cc1S(=O)(=O)NC1CC1)N1CCOCC1 ; NC(=O)c1cc(S(=O)(=O)Nc2ccc(O)cc2)cs1 ; COc1cc(NC(=O)C2CC(=O)N=C(N)S2)c(OC)cc1Cl ; Cc1c(Cl)cccc1NC(=O)c1cccc(NS(C)(=O)=O)c1]

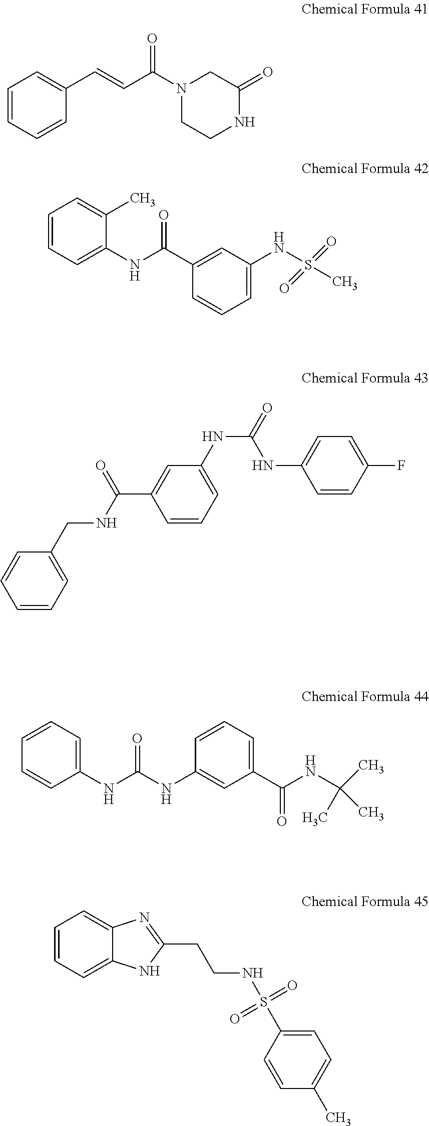 [SMILES: O=C1CN(C(=O)/C=C/c2ccccc2)CCN1 ; Cc1ccccc1NC(=O)c1cccc(NS(C)(=O)=O)c1 ; O=C(Nc1ccc(F)cc1)Nc1cccc(C(=O)NCc2ccccc2)c1 ; CC(C)(C)NC(=O)c1cccc(NC(=O)Nc2ccccc2)c1 ; Cc1ccc(S(=O)(=O)NCCc2nc3ccccc3[nH]2)cc1]

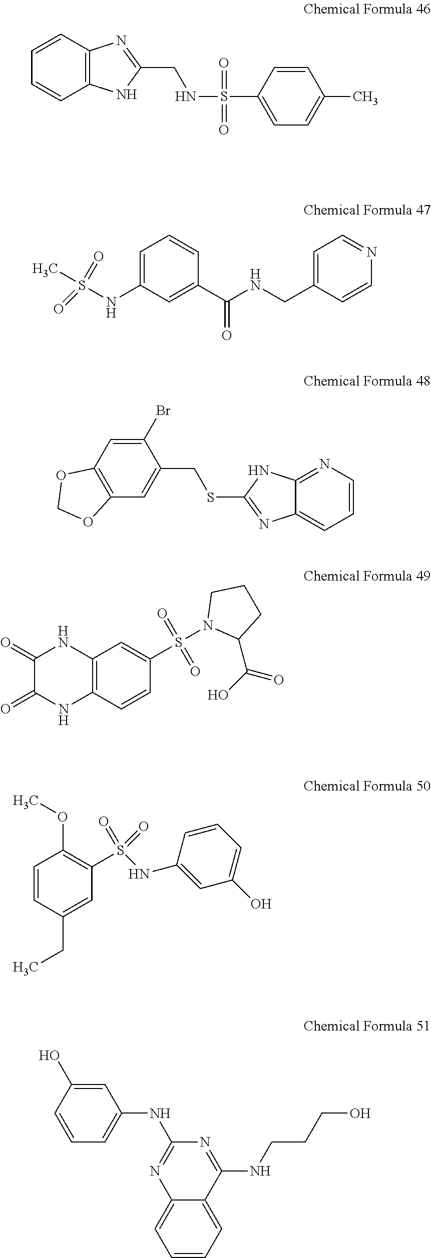

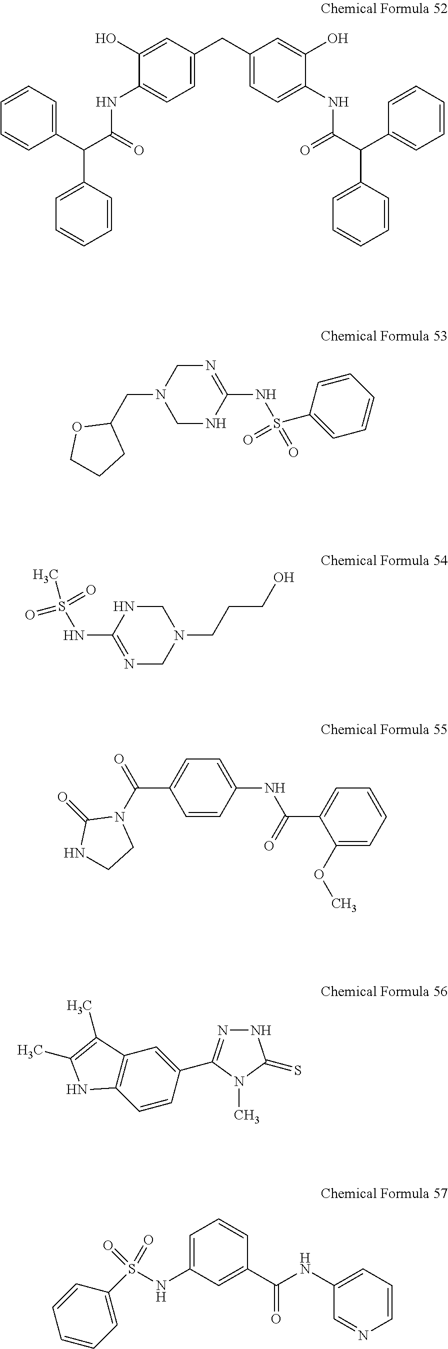 [SMILES: O=C(Nc1ccc(Cc2ccc(NC(=O)C(c3ccccc3)c3ccccc3)c(O)c2)cc1O)C(c1ccccc1)c1ccccc1 ; O=S(=O)(NC1=NCN(CC2CCCO2)CN1)c1ccccc1 ; CS(=O)(=O)NC1=NCN(CCCO)CN1 ; COc1ccccc1C(=O)Nc1ccc(C(=O)N2CCNC2=O)cc1 ; Cc1[nH]c2ccc(-c3n[nH]c(=S)n3C)cc2c1C ; O=C(Nc1cccnc1)c1cccc(NS(=O)(=O)c2ccccc2)c1]

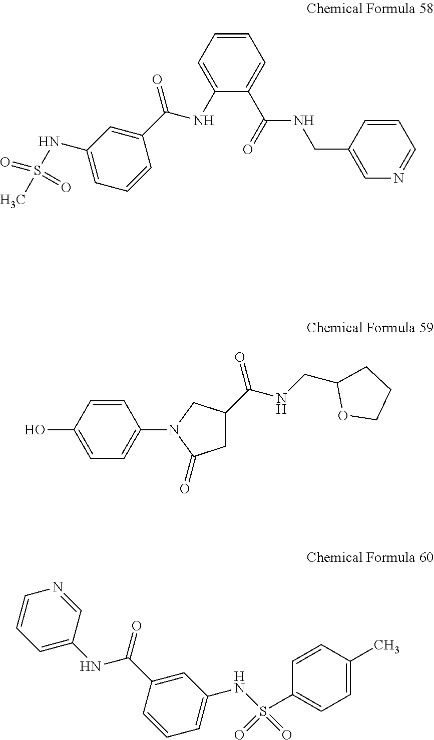 [SMILES: CS(=O)(=O)Nc1cccc(C(=O)Nc2ccccc2C(=O)NCc2cccnc2)c1 ; O=C(NCC1CCCO1)C1CC(=O)N(c2ccc(O)cc2)C1 ; Cc1ccc(S(=O)(=O)Nc2cccc(C(=O)Nc3cccnc3)c2)cc1]

The present inventors have made extensive efforts to develop novel compounds exhibiting selective STAT3-inhibitory activity for the prevention and treatment of various diseases including cancer, autoimmune diseases and inflammatory diseases caused by excessive expression or activation of STAT3. As a result, the present inventors have confirmed that derivatives having 3-phenoxymethyl-1,2,4-oxadiazole or 3-phenoxymethyl-1,2,4-thiadiazole as a skeletal structure are effective in STAT3-related diseases since the derivatives inhibit activation of STAT3, that is, inhibit the phosphorylation thereof to activate caspase-3 and PARP, inhibit the activity of MMPs, and thus effectively inhibit the expression of Twist gene. Consequently, the substances which efficiently inhibit the activity of STAT3 of the present invention can be an efficient prophylactic agent and an efficient therapeutic agent for various STAT3-related diseases such as cancer, diabetic retinopathy, diabetes, autoimmune diseases and inflammatory diseases.

The present invention includes not only compounds selected from the group consisting of compounds represented by Chemical Formulas 1 to 60 but also pharmaceutically acceptable salts, solvates and hydrates thereof.

As used herein, the term “pharmaceutically acceptable salt” refers to a formulation of a compound that does not cause serious irritation to the organism to which the compound is administered and does not impair the biological activity and properties of the compound. The pharmaceutical salts can be obtained by reacting the compounds of the present invention with inorganic acids such as hydrochloric acid, bromic acid, sulfuric acid, nitric acid, and phosphoric acid, sulfonic acids such as methanesulfonic acid, ethanesulfonic acid, and p-toluenesulfonic acid, and organic carboxylic acids such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid, and salicylic acid. Moreover, the pharmaceutical salts can also be obtained by reacting the compounds of the present invention with bases to form salts such as ammonium salts, alkali metal salts such as, sodium or potassium salt, and alkaline earth metal salts such as calcium or magnesium salts, salts of organic bases such as dicyclohexylamine, N-methyl-D-glucamine, and tris(hydroxymethyl)methylamine, and amino acid salts such as arginine and lysine.

As used herein, the term “hydrate” refers to a compound of the present invention or a salt thereof containing a stoichiometric or non-stoichiometric amount of water bound by non-covalent intermolecular force. The term “solvate” refers to a compound of the present invention or a salt thereof containing a stoichiometric or non-stoichiometric amount of solvent bound by non-covalent intermolecular force. Preferred solvents for this are volatile solvents, non-toxic solvents and/or solvents suitable for administration to humans.

According to another aspect, the present invention provides a pharmaceutical composition for prevention or treatment of STAT3-related diseases containing (a) a pharmaceutically effective amount of a compound selected from the group consisting of compounds represented by Chemical Formulas 1 to 60 above, or a pharmaceutically acceptable salt, solvate or hydrate thereof; and (b) a pharmaceutically acceptable carrier.

As used herein, the term “prevention” means all actions to inhibit STAT3-related diseases or delay the progression of STAT3-related diseases by administration of the composition of the present invention. The term “treatment” means (i) inhibition of development of STAT3-related diseases, (ii) alleviation of the diseases, and (iii) elimination of the diseases.

As used herein, the term “STAT3-related diseases” refers to diseases of which the onset, progression, prognosis or treatment sensitivity is directly or indirectly affected by the overexpression and/or overactivation of STAT3. The STAT3-related diseases include cancer, diabetic retinopathy, diabetes, hemophilic arthrosis, atherosclerosis, keloid, wound granulation, vascular adhesion, autoimmune diseases, restenosis, intestinal adhesions, cat scratch diseases, ulcers, cirrhosis, diabetic nephropathy, malignant neurosis, thrombotic microangiopathy, organ transplant rejection, glomerulopathy, neurodegenerative diseases and inflammatory diseases.

According to an embodiment of the present invention, the cancer is selected from the group consisting of gastric cancer, colorectal cancer, lung cancer, human malignant breast cancer, ovarian cancer, liver cancer, bronchial cancer, nasopharyngeal cancer, laryngeal cancer, pancreatic cancer, bladder cancer, colon cancer, cervical cancer, brain cancer, prostate cancer, bone cancer, skin cancer, thyroid cancer, leukemia, lymphoma, adrenal cortical cancer, parathyroid cancer, ureteric cancer, glioma, esophageal cancer, small intestine cancer, glioblastoma, brain tumor and kidney cancer.

According to an embodiment of the present invention, the autoimmune diseases are selected from the group consisting of alopecia greata, ankylosing spondylitis, antiphospholipid syndrome, autoimmune Addison disease, adrenal autoimmune disease, autoimmune hemolytic anemia, autoimmune hepatitis, autoimmune ovaryitis and orchitis, autoimmune thrombocytopenia, Behcet's disease, vesicular ichthyosis, cardiomyopathy, celiac sprue-dermatitis, chronic fatigue immunodeficiency syndrome, chronic inflammatory demyelinating multiple neuropathy, Chur-strauss syndrome, reflexive Yucheonpochang, CREST syndrome, cold agglutinin disease, Crohn's disease, discous lupus, abdominal complex cold globulinemia, fibromyalgia-fibromyositis, glomerulonephritis, Graves' disease, Schwain-Barre syndrome, Hashimoto's thyroiditis, idiopathic pulmonary fibrosis, idiopathic thrombocytopenic purpura, IgA neuritis, combustive arthritis, lichen planus, lupus erythematosus, Menirre's disease, mixed connective tissue disease, multiple sclerosis, type I or immune-mediated diabetes, myasthenia gravis, vulgar astrocyst, malignant anemia, crystalline polyarteritis, Badal-chondritis, autoimmune polyline syndrome, rheumatoid multiple myalgia, multiple myositis and dermatomyositis, primary agamma globulinemia, primary glycemic cirrhosis, psoriasis, psoriatic arthritis, Raynaud's phenomenon, Reiter's syndrome, rheumatoid arthritis, sarcoidosis, scleroderma, ankylosing human syndrome, systemic lupus erythematosus, lupus erythematosus, Takayasu's arteritis, transient arteritis, giant cell arteritis, ulcerative colitis, uveitis, vitiligo, and Wegener's granulomatosis.

According to an embodiment of the present invention, the inflammatory diseases are selected from the group consisting of asthma, encephalitis, inflammatory enteritis, rheumatoid arthritis, chronic obstructive pulmonary disease, allergic, atopic dermatitis, psoriasis, pulmonary thrombosis, pefiosis, undifferentiated spinal joint disease, Crohn's disease, pancreatitis, dermatitis, undifferentiated arthrosis, arthritis, glomerulonephritis, bronchitis, inflammatory osteolysis, and chronic inflammation caused by viral or bacterial infection.

According to an embodiment of the present invention, the compounds represented by Chemical Formulas 1 to 60 of the present invention selectively inhibit the activity of STAT3 and inhibit the metastasis of cancer cells. As confirmed in the following Examples, the compounds of the present invention effectively activate caspase-3 and PARP and inhibit the activity of MMPs and the expression of Twist gene, and thus efficiently inhibit the growth and metastasis of human tumor cells. More specifically, the compounds of the present invention inhibit phosphorylation of tyrosine 705 residues and serine 727 residues of STAT3, thus inhibit the STAT3 signaling pathway, STAT3 dimer migration to the nucleus, inhibit the proliferation of cancer cells caused by the inhibition of caspase-3 and PARP activity, and inhibit the cancer cell death inhibition mechanism and the migration and invasion of cancer cells and growth and metastasis of tumors induced by the inhibition of MMPs activity and the expression of Twist gene.

The composition of the present invention contains a pharmaceutically acceptable carrier. The pharmaceutically acceptable carrier contained in the composition of the present invention is one commonly used in formulation and includes, but is not limited to, lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil and the like. The pharmaceutical composition of the present invention may further contain a lubricant, a wetting agent, a sweetener, a flavoring agent, an emulsifying agent, a suspending agent, a preservative and the like in addition to the above ingredients. Suitable pharmaceutically acceptable carriers and formulations are described in detail in Remington's Pharmaceutical Sciences (19th ed., 1995).

The pharmaceutical composition of the present invention can be administered orally or parenterally and may be administered by intravenous injection, subcutaneous injection, intramuscular injection, intraperitoneal injection, transdermal administration, and the like in the case of parenteral administration.

The suitable dosage of the pharmaceutical composition of the present invention may be variously prescribed by factors such as formulation method, mode of administration, and the patient's age, weight, sex, morbidity, food, time of administration, route of administration, rate of excretion, and response sensitivity. The daily dosage of the pharmaceutical composition of the present invention is, for example, 0.0001 to 100 mg/kg.

The pharmaceutical composition of the present invention may be prepared in unit dose form by being formulated with a pharmaceutically acceptable carrier and/or excipient or may be prepared to be incorporated into a multi-dose container according to a method that can be easily carried out by those skilled in the art to which the present invention pertains. At this time, the formulation is in the form of a solution, suspension, syrup or emulsion in an oil or aqueous medium, or may be in the form of ex-agent, powdery remedy, powder, granule, tablet or capsule, and may additionally contain a dispersing agent or a stabilizing agent.

According to another aspect, the present invention provides a food composition for prevention or improvement of STAT3-related diseases containing a compound selected from the group consisting of compounds represented by Chemical Formulas 1 to 60 above or a salt thereof as an active ingredient.

The food composition is not particularly limited, but may be all forms of food such as health functional food, nutritional supplement, nutritional supplement, pharmafood, health food, nutraceutical, designer food, and food additives. Examples thereof include meat, sausage, bread, chocolate, candy, snacks, confectionery, pizza, ramen, other noodles, gums, dairy products including ice creams, various soups, beverages, teas, drinks, alcoholic beverages, and vitamin complexes.

The food composition of the present invention contains ingredients to be commonly added during food production and contains, for example, proteins, carbohydrates, fats, nutrients, seasoning and flavoring agents. Examples of the above-mentioned carbohydrates include monosaccharides such as glucose and fructose; disaccharides such as maltose, sucrose, and oligosaccharides; and polysaccharides, for example, conventional sugars such as dextrin and cyclodextrin, and sugar alcohols such as xylitol, sorbitol, and erythritol. As flavoring agents, natural flavoring agents (Tau Martin, Stevia extract (for example, rebaudioside A and glycyrrhizine)) and synthetic flavoring agents (saccharin, aspartame and the like) can be used.

In addition to the above, the food composition of the present invention may contain various nutrients, vitamins, minerals (electrolytes), dietary ingredients, flavoring agents such as synthetic flavoring agents and natural flavoring agents, coloring agents and neutralizing agents (cheese, chocolate and the like), pectic acid and salts thereof, alginic acid and salts thereof, organic acids, protective colloidal thickeners, pH adjusting agents, stabilizers, preservatives, glycerin, alcohols, carbonating agents used in carbonated drinks, and the like. For example, when the food composition of the present invention is prepared as a drink, the food composition may further contain citric acid, liquid fructose, sugar, glucose, acetic acid, malic acid, fruit juice, various plant extracts and the like in addition to the active ingredient of the present invention.

As another aspect, the present invention provides a cosmetic composition (functional cosmetic composition) for prevention or improvement of STAT3-related diseases containing a compound selected from the group consisting of compounds represented by Chemical Formulas 1 to 60 above or a salt thereof as an active ingredient.

The cosmetic composition may be prepared in any formulation conventionally prepared in the art, contains ingredients commonly used in the manufacture of cosmetics in addition to the active ingredient, and may contain, for example, conventional adjuvants such as an antioxidant, a stabilizer, a solubilizer, vitamins, a pigment and a fragrance.

As another aspect, the present invention provides a method of preventing or treating STAT3-related diseases including administering a compound selected from the group consisting of compounds represented by Chemical Formulas 1 to 60 above or a salt thereof to an individual in a pharmaceutically effective amount.

As another aspect, the present invention provides the use of a compound selected from the group consisting of compounds represented by Chemical Formulas 1 to 60 above or a salt thereof to be used in a pharmaceutical composition for prevention or treatment of STAT3-related diseases.

As another aspect, the present invention provides the use of a compound selected from the group consisting of compounds represented by Chemical Formulas 1 to 60 above or a salt thereof to be used in a food composition for prevention or improvement of STAT3-related diseases.

As another aspect, the present invention provides the use of a compound selected from the group consisting of compounds represented by Chemical Formulas 1 to 60 above or a salt thereof to be used in a cosmetic composition (functional cosmetic composition) for prevention or improvement of STAT3-related diseases.

### Advantageous Effects of Invention

The features and advantages of the present invention are summarized as follows:

(i) The present invention provides novel compounds exhibiting STAT3 inhibitory activity and uses thereof.

(ii) Pharmaceuticals, cosmeceuticals, cosmetics, neutraceuticals or food compositions can be prepared using the compounds of the present invention as an active ingredient.

(iii) The compounds of the present invention efficiently inhibit the abnormal activity of STAT3 associated with various diseases and thus can be usefully utilized for the prevention and treatment of various STAT3-related diseases associated with cancer, autoimmune diseases, inflammatory diseases, and the like.

### BRIEF DESCRIPTION OF DRAWINGS

FIGS. 1A to 1G illustrate simulation utilizing cancer cells derived from Drosophila and humans and a binding-molecular structure model for an SH2 domain to which STAT3 binds for showing that the ODZ17690 compound of the present invention exhibits selective activity on STAT3.

FIGS. 2A and 2B show that the ODZ17690 compound of the present invention exerts an excellent action of selectively inhibiting the activity of STAT3 in a human-derived Hodgkin lymph cancer cell line (L540 cells).

FIGS. 3A to 3C show 13 substances having excellent activity for inhibiting STAT3 among 144 substances having 3-phenoxymethyl-1,2,4-oxadiazole or 3-phenoxymethyl-1,2,4-thiadiazole as a skeletal structure.

FIGS. 4A to 4H show the STAT3 inhibitory action of compound ODZ10117 in a binding-molecular structure model for a SH2 domain to which STAT3 binds and in various human-derived cancer cells.

FIGS. 5A to 5C show that the ODZ10117 compound inhibits STAT3 activity in human malignant glioblastoma (U87-MG) cells and human malignant breast cancer (MDA-MB-231) cells.

FIGS. 6A and 6B show that the ODZ10117 compound inhibits the activity of STAT92E (mammal STAT) in Drosophila cells.

FIGS. 7A to 7D show that the ODZ10117 compound inhibits the activity of STAT3 in various kinds of human malignant glioblastoma.

FIGS. 8A to 8D show that the ODZ10117 compound inhibits the activity of STAT3 in various kinds of human malignant breast cancer cells.

FIGS. 9A to 9D show that the ODZ10117 compound inhibits dimerization, nuclear translocation and transcriptional activity of STAT3.

FIG. 10 shows that the ODZ10117 compound inhibits STAT3-dependent cell survival in human malignant glioblastoma.

FIGS. 11A to 11E show that the ODZ10117 compound inhibits the proliferation of U87-MG cells and MDA-MB-231 cells and induces apoptosis.

FIGS. 12A to 12D show that the ODZ10117 compound inhibits the migration and invasion of U87-MG cells and MDA-MB-231 cells.

FIGS. 13A to 13D show that the ODZ10117 compound inhibits the size of growing tumor in xenograft mice.

FIGS. 14A to 14D show STAT3 inhibitory activity of 46 compounds presented in [Table 1] of the present invention in a human Hodgkin lymphoma cancer cell line.

FIGS. 15A and 15B show the STAT3 inhibitory activity of 33 compounds which exert remarkably excellent STAT3 inhibitory activity in a human Hodgkin lymphoma cancer cell line among 46 compounds presented in [Table 1] of the present invention.

FIG. 16 shows the STAT3 inhibitory activity of two compounds which exert extremely excellent STAT3 inhibitory activity among 46 compounds presented in [Table 1] of the present invention.

### DESCRIPTION OF EMBODIMENTS

Hereinafter, the present invention will be described in more detail with reference to Examples. These Examples are only intended to illustrate the present invention in more detail, and it will be apparent to those skilled in the art that the scope of the present invention is not limited by these Examples according to the gist of the present invention.

### EXAMPLES

### Experimental Materials and Methods

### Material Samples

5-tert-butyl-3-(3-nitro-phenoxymethyl)-[1,2,4]oxadiazole (ODZ17690), 3-(2,4-dichloro-phenoxymethyl)-5-trichloromethyl-[1,2,4]oxadiazole (ODZ10117), 5-sec-butoxy-3-(2,4-dichloro-phenoxymethyl)-[1,2,4]oxadiazole (ODZ8292), 3-(2,4-dichloro-phenoxymethyl)-5-isobutoxy-[1,2,4]oxadiazole (ODZ8293), 3-(2,4-dichloro-phenoxymethyl)-5-propoxy-[1,2,4]oxadiazole (ODZ10181), 3-(2,4-dichloro-phenoxymethyl)-5-prop-2-ynyloxy-[1,2,4]oxadiazole (ODZ8297), 5-allyloxy-3-(4-chloro-2-methyl-phenoxymethyl)-[1,2,4]oxadiazole (ODZ8315), 3-(2,4-dichloro-phenoxymethyl)-[1,2,4]oxadiazole-5-carboxylic acid methylamide (ODZ10159), 3-(2,4-dichloro-phenoxymethyl)-5-ethoxy-[1,2,4]oxadiazole (ODZ10177), 1-{4-chloro-5-[5-(2,4-dichloro-phenyl)-[1,2,4]oxadiazol-3-ylmethoxy]-2-fluoro-phenyl}-3,4-dimethyl-pyrrole-2,5-dione (ODZ11296), 3,6-dichloro-8-(3-m-toyloxymethyl-[1,2,4]oxadiazol-5-yl)-quinoline (ODZ16717), 1-phenyl-3-[2-(3-m-toyloxymethyl-[1,2,4]oxadiazol-5-yl)-phenyl]-urea (ODZ16998), 3-chloro-2-(3-m-toyloxymethyl-[1,2,4]oxadiazol-5-yl)-phenylamine (ODZ16999) that have 3-phenoxymethyl-1,2,4-oxadiazole as a skeletal structure and 2-{2-[3-(2-chloro-4-fluoro-phenoxymethyl)-[1,2,4]thiadiazol-5-yloxymethyl]-phenyl}-2-methoxyimino-N-methyl-acetamide (ODZ9562) having 3-phenoxymethyl-1,2,4-thiadiazole as a skeletal structure were prepared by the following methods, suspended in 100% dimethyl sulfoxide (DMSO), and stored at −20° C. to be used.

### Synthesis of Compounds

### Preparation Example 1

### Preparation of 3-((2,4-dichlorophenoxy)methyl)-5-(trichloromethyl)-1,2,4-oxadiazole (ODZ10117)

### <1-1> Preparation of 2-(2,4-dichlorophenoxy) acetonitrile

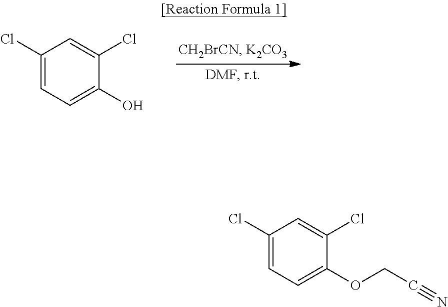 [SMILES: Oc1ccc(Cl)cc1Cl ; N#CCOc1ccc(Cl)cc1Cl]

After 2,4-dichlorophenol (1 g, 6.10 mmol) was dissolved in dimethylformamide (8 mL), potassium carbonate (840 mg, 6.10 mmol) was added thereto at room temperature, and then bromoacetonitrile (0.44 mL, 6.10 mmol) dissolved in dimethylformamide (3 mL) was slowly dropped to the mixture. After the resultant mixture was stirred at room temperature for 24 hours, water was added to the reaction mixture, and extraction with ethyl acetate was performed. Thereafter, the organic layer was washed with a saturated aqueous solution of sodium sulfate and dried over magnesium sulfate, then the solvent was removed under reduced pressure, and then the residue was subjected to purification by silica gel column chromatography (n-hexane:ethyl acetate=3:1) was performed to obtain the desired compound (1.18 g, 95%).

### <1-2> Preparation of 2-(2,4-dichlorophenoxy)-N′-hydroxyacetimidamide

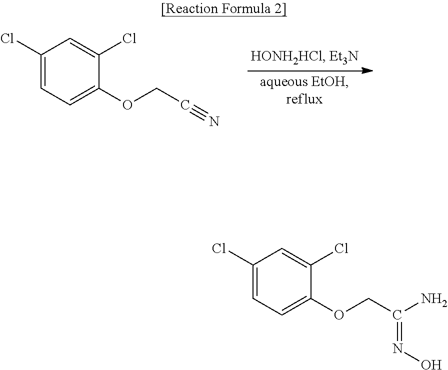 [SMILES: N#CCOc1ccc(Cl)cc1Cl ; N/C(COc1ccc(Cl)cc1Cl)=N\O]

Triethylamine (2.70 ml, 19.52 mmol) was dissolved in 50% ethanol aqueous solution (21 ml), and then hydroxylamine hydrochloride (1.36 g, 19.52 mmol) was added thereto at room temperature. After the mixture was stirred at room temperature for 5 minutes, 2-(2,4-dichlorophenoxy)acetonitrile obtained was dissolved in ethanol (83 ml) and added to the resultant mixture, and reflux was performed at 100° C. for 3 hours. Thereafter, the reaction mixture was diluted with water, filtered, and washed again with water to obtain the desired compound (2.71 g, 77%). 1H NMR (500 MHz, CDCl3) δ4.58 (s, 2H), 4.85 (s, 2H), 6.53 (s, 1H), 6.96 (d, J=8.8 Hz, 1H), 7.17 (dd, J=2.3 Hz, 8.8 Hz, 1H), 7.37 (d, J=2.4 Hz, 1H)

### <1-3> Preparation of 3-((2,4-dichlorophenoxy)methyl-5-(trichloromethyl)-1,2,4-oxadiazole (ODZ10117)

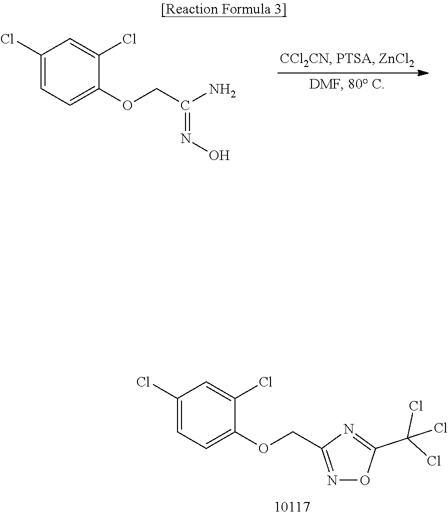 [SMILES: N/C(COc1ccc(Cl)cc1Cl)=N\O ; Clc1ccc(OCc2noc(C(Cl)(Cl)Cl)n2)c(Cl)c1]

2-(2,4-Dichlorophenoxy)-N′-hydroxyacetimidamide (100 mg, 0.43 mmol) obtained and trichloroacetonitrile (0.040 mL, 0.43 mmol) were dissolved in dimethylformamide (1 mL), and then para-toluene sulfonic acid (41 mg, 0.22 mmol) and zinc chloride (30 mg, 0.22 mmol) were added thereto. Thereafter, the mixture was refluxed at 80° C. for 16 hours, and the organic layer of the reaction compound obtained was washed with sodium bicarbonate and dried over magnesium sulfate, and the solvent was removed under reduced pressure. Subsequently, the remaining residue was subjected to purification by silica gel column chromatography (n-hexane:ethyl acetate=10:1) to obtain the desired compound (81 mg, 53%). 1H NMR (300 MHz, CDCl3) δ5.27 (s, 2H), 7.01 (d, J=8.8 Hz, 1H),7.20 (dd, J=2.5 Hz, 8.7 Hz, 1H),7.40 (d, J=2.6 Hz, 1H)

### Preparation Example 2

### Preparation of 3-((2,4-dichlorophenoxy)methyl)-5-propoxy-1,2,4-oxadiazole (ODZ10181)

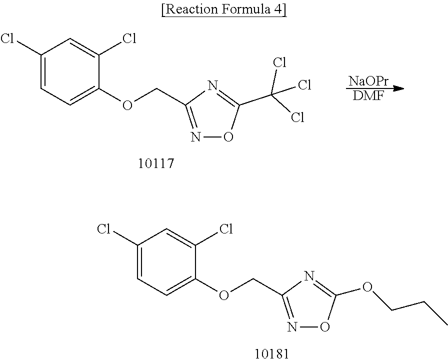

After sodium propoxide (119 mg, 1.45 mmol) was dissolved in dimethylformamide (10 mL), 3-((2,4-dichlorophenoxy)methyl-5-(trichloromethyl)-1,2,4-oxadiazole (350 mg, 0.97 mmol) obtained was added thereto at room temperature, and then the mixture was stirred at room temperature for 10 minutes. The reaction mixture was washed with saturated aqueous sodium sulfate solution and saline solution, and then the organic layer was dried over magnesium sulfate. Thereafter, the solvent was removed under reduced pressure, and the residue was subjected to purification by silica gel column chromatography (n-hexane:ethyl acetate=10:1) to obtain the desired compound (64 mg, 22%). 1H NMR (400 MHz, CDCl3) δ1.02 (t, J=7.4 Hz, 3H), 1.85 (qd, J=7.0 Hz, 14.1 Hz, 2H), 4.47 (t, J=6.6 Hz, 2H), 5.05 (s, 2H), 7.00 (d, J=8.8 Hz, 1H), 7.17 (dd, J=2.0 Hz, 8.8 Hz, 1H) , 7.37 (d, J=1.9 Hz, 1H)

### Preparation Example 3

### Preparation of 5-(sec-butoxy)-3-((2,4-dichlorophenoxy)methyl)-1,2,4-oxadiazole (ODZ8292)

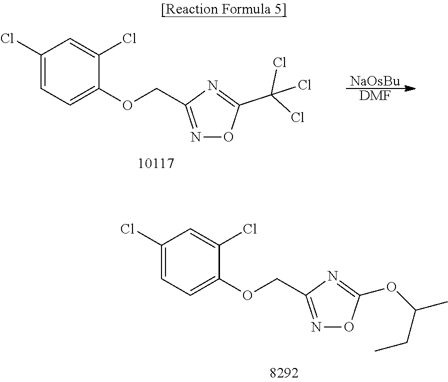

After sodium secondary butoxide (613 mg, 6.37 mmol) was dissolved in dimethylformamide (20 mL), 3-((2,4-dichlorophenoxy)methyl-5-(trichloromethyl)-1,2,4-oxadiazole (462 mg, 1.27 mmol) obtained was added thereto at room temperature, and the mixture was stirred at room temperature for 10 minutes. Thereafter, the reaction mixture obtained was washed with saturated aqueous sodium sulfate solution and saline solution, and then the organic layer extracted with ethyl acetate was dried over magnesium sulfate. Next, the solvent was removed under reduced pressure, and the residue was subjected to purification by silica gel column chromatography (n-hexane:ethyl acetate=15:1) to obtain the desired compound (78 mg, 19%). 1H NMR (400 MHz, CDCl3) δ0.97 (t, J=7.4 Hz, 3H), 1.42 (d, J=6.2 Hz, 3H), 1.67-1.87 (m, 2H), 5.05 (s, 2H), 7.00 (d, J=8.8 Hz, 1H), 7.17 (dd, J=2.0 Hz, 8.8 Hz, 1H), 7.37 (d, J=2.0 Hz, 1H)

### Structure-Based Virtual Screening

AutoDock version 4.2 software was used to screen for compounds which inhibit STAT3 activity, and virtual screening was performed using a compound library from ChemBridg (https://www.hit2lead.com/). In the X-ray crystal structure of STAT3 (PDB ID: 1BG1), a three-dimensional structure of the portion corresponding to the SH2 domain was used (Becker S, et al. Nature. 394, 145-151 (1998)). The minimum energy was calculated using the AMBER package (ver. 11) (Case D A, et al. J Comput Chem. 26, 1668-1688 (2005)). In addition, a structure was created for the screening compound of ChgemBridge along with the AMBER program, and a molecular dynamics simulation was performed. All conformers were docked to the SH domain by the AutoDock package (ver 4.2) with basic default parameters. In order to dock the protein-ligand interaction through the GB/SA model, all docked structures were generated using a molecular dynamics simulation using AMBER force field. The resulting structure was clustered based on the structural similarity, and the structure belonging to the cluster representing the lowest score function was selected. Considering the entropy effect for the binding energy as well, the central conformer structure and energy value of the cluster were selected as representative ones. All processes were automatically executed by the ALIS-DOCK (Automatic cLuster-used Iterative Structure-based DOCKing) script. All calculations were performed using a Mac mini based cluster system.

### Drosophila Cell Culture and Luciferase Assay

Drosophila cells (S2-NP, 24×3) used in the experiment were cultured as previously described (Kim B H, et al. Mol Cancer Ther. 7, 2672-2680 (2008)). S2-NP (Drosophila Schneider cell) cells were Drosophila-derived macrophage-like cells and were cultured in Schneider's medium. 24×3 cells were cell lines stably expressed by inserting 10XSTAT92E-luciferase and PolIII-Renilla luciferase-labeled plasmid DNA as reporter constructs into S2-NP cells and were cultured in a medium prepared by adding 500 mm g/ml of geneticin to the same medium as that for S2-NP. S2-NP cells in which unpaired (upd) or HOPT^{Tum-l} as a ligand was expressed and 24×3 cells constructed with reporter constructs were mixed at a constant ratio and cultured for 24 hours in the presence of a compound, the expression level of STAT92E was measured using a luminometer, and the inhibition effect was compared with that of the DMSO-treated group. In addition, the inhibitory effect was compared with that of the AG490, nifuroxazide, NSC628869 (STA-21 or STA) or NSC74859 (S31-001)-treated group as a positive control.

### Human Cancer Cell Culture

The human cancer cell lines used in the experiment were U87-MG (human malignant glioblastoma) and MDA-MB-231 (human malignant breast cancer) cell lines, and various kinds of human-derived blood and solid cancer cells including these human cancer cell lines were cultured as previously described (Jung J E, et al. FASEB J. 19, 1296-1298 (2005)). The human Hodgkin lymphoma cancer cell lines L540 and HLDM-2 used in the experiment were obtained from the German Collection of Microorganism and Cell Cultures (DSMZ, Germany) and cultured using RPMI 1640 medium containing 20% FBS in a 5% CO₂ incubator at a temperature of 37° C.

### Binding-Molecular Model Design

In the X-ray crystal structure of STAT3 (PDB ID: 1BG1), a three-dimensional structure of the portion corresponding to the SH2 domain was used (Becker S, et al. Nature. 394, 145-151 (1998)). The minimum energy was calculated using the AMBER package (ver. 11) (Case D A, et al. J Comput Chem. 26, 1668-1688 (2005)). In addition to the AMBER program, 500 structures were generated for all the compounds including ODZ10117, and molecular dynamics simulation was performed.

### Cell Proliferation Assay

Cell proliferation assay was performed as previously described (Won C, et al. Anticancer Res. 30, 481-488 (2010)). In order to analyze the cell proliferation rate, U87-MG and MDA-MB-231 cell lines were dispensed into a 6-well culture dish, then treated with DMSO and ODZ10117 the next day, and stained with crystal violet after 0 h, 24 h, 48 h, and 72 h, and the number of live cells was calculated using a hemocytometer.

### Transformation and Luciferase Assay

Assay on the expression of luciferase was performed as previously described (Jung J E, et al. FASEB J. 19, 1296-1298 (2005)). Plasmid DNA having STAT3-TA-luciferase as a reporter construct was temporarily expressed in HEK293T cells using Lipofectamine 2000, the HEK293T cells were treated with ODZ10117, the level of STAT3 expression was measured using a luminometer, and the inhibitory effect was compared with that of the DMSO-treated group. In addition, the inhibitory effect was compared with that of the AG490, nifuroxazide, NSC628869 (STA-21 or STA) or NSC74859 (S31-001)-treated group as a positive control.

### Real-Time Polymerase Chain Reaction (qRT-PCR)

RNA isolation and real-time polymerase chain reaction were performed as described previously (Jung J E, et al. FASEB J. 19, 1296-1298 (2005)). Primer specific for human-derived Bcl-XL, Bcl-2, Twist, and GADPH genes were mixed with QuantiFast SYBR Green PCR master mix, and each gene was amplified using the Applied Biosystems 7300 real-time PCR system to compare the expression level.

### Western Blot Analysis

Western blot analysis was performed (Jung J E, et al. FASEB J. 19, 1296-1298 (2005)). The cells were washed two times with PBS and suspended in the lysate (50 mM Tris-HCl, pH 7.4, 350 mM NaCl, 1% Triton X-100, 0.5% Nonidet P-40, 10% glycerol, 0.1% SDS, 1 mM EDTA, 1 mM EGTA, 1 mM Na3VO4, 1 mM PMSF, protease and phosphorylation inhibitor) and the cell membrane and nuclear membrane were crushed to lyse the cells. The insoluble protein fraction was removed by centrifugation, and the supernatant was mixed with SDS electrophoresis buffer and electrophoresis (SDS-PAGE) was performed to detect the protein with the desired antibody and to confirm the protein expression level.

### Wound Healing Assay

Wound healing assay was performed as previously described (Jung J E, et al. FASEB J. 19, 1296-1298 (2005)). The respective cells were dispensed into a 12-well plate (U87-MG: 5×10⁵ cells/ml; MDA-MB-231: 3×10⁵ cells/ml) and cultured until the cells proliferated 90% or more. Cells scraped off from the plate with the tip of the pipette were washed two times with PBS. After 24 hours, the proliferation (invasion) of cells at the part from which the cells were had been removed was observed under a microscope, and images were taken.

### Matrigel Invasion Analysis

The growth factor-reduced Matrigel was diluted with a serum-free culture solution at a 1:3 ratio, transferred to a 24-well plate, and solidified at 37° C. for 5 hours. The cells were placed in a culture solution containing 1% serum and added to the solidified Matrigel, 600 μl of a culture solution containing 10% serum to which fibronectin was added at a concentration of 5 μl/ml was added thereto, and the cells were cultured for 24 hours. Thereafter, the cells were fixed and stained by adding Diffquick solution thereto and then observed under a Leica Application Suite microscope, and images were taken.

### Flow-Cytometry

In order to analyze the degree of apoptosis, the respective cells were washed with FACS buffer and centrifuged for 2 minutes at 2000 rpm. The washed cells were stained with propidium iodide for 15 minutes and analyzed by FACS Canto flow cytometry and then Flow-Jo software.

### Xenograft of Human Tumor Cell

Male Nude Mouse (BALB/cAnNCrj-nu/nu) was purchased from Charles River Japan Inc. (Shin-Yokohama, Japan). The mouse was bred in a clean room in which the temperature and humidity were constantly controlled. The breeding process was carried out according to the method described in the manual for maintenance of laboratory animals in Seoul National University. U87-MG cells or MDA-MB-231 cells were diluted with PBS, suspended in 100 μl of Matrigel at 25% concentration, and injected into the back of mouse and bred for 6 to 10 weeks. Human tumor growth was measured every other day using a vernier caliper from 2 weeks after cell injection. With regard to the degree of inhibition of human tumor cell formation, 40 μM of ODZ10117 was directly injected into the human tumor on days 0, 3, and 5 from week 4 of tumor cell injection. On the 7th day, the mouse was sacrificed to separate the tumor, and the tumor cell inhibitory efficacy of the drug was measured by being compared with that of the control group treated with 1% DMSO.

### Tumor Histology and Immunostaining of p-STAT3, Cleaved Caspase-3, Bcl-XL, Ki67 and MMP2

Tumors isolated from mice were fixed with formalin and embedded with paraffin, and sections were cut from each paraffin block. For histological evaluation, the sections were stained with hematoxylin-eosin, phospho-STAT3, cleaved caspase-3, Bcl-XL, Ki67, and pro/active MMP-2, respectively. The paraffin of the sections was removed, moisture was removed using alcohol, and then the sections were heated in 10 mM sodium citrate buffer (pH: 6.0) for 5 minutes with microwaves. Non-specific binding was removed by reacting the sections with a PBS solution containing 2.5% bovine serum albumin and 2% normal goat serum for 1 hour. The sections were reacted with antibodies against phospho-STAT3, cleaved caspase-3, Bcl-XL, Ki67, and pro/active MMP-2 diluted 1:100 overnight at 4° C. As a negative control, the sections were reacted with a dilution solution in a state of not containing primary antibodies. The sections were then washed and reacted with secondary antibodies labeled with appropriate biotin, and the location and expression of the bound antibodies were confirmed using an avidin-biotin-horseradish complex. For histological evaluation, each stained section was observed under a microscope at 100-fold and 400-fold magnifications and analysis was performed using a Sony XC-77 CCD camera and a microcomputer imaging device Model 4 image analysis system.

### Statistical Analysis

All data were analyzed using Microsoft Excel 2000 software. Data were expressed as mean values and standard errors, and statistical significance was calculated by unpaired two-tailed Student's t-test (p<0.05).

### Experiment Results

### Identification of ODZ17690 as selective STAT3 Inhibitory Precursor

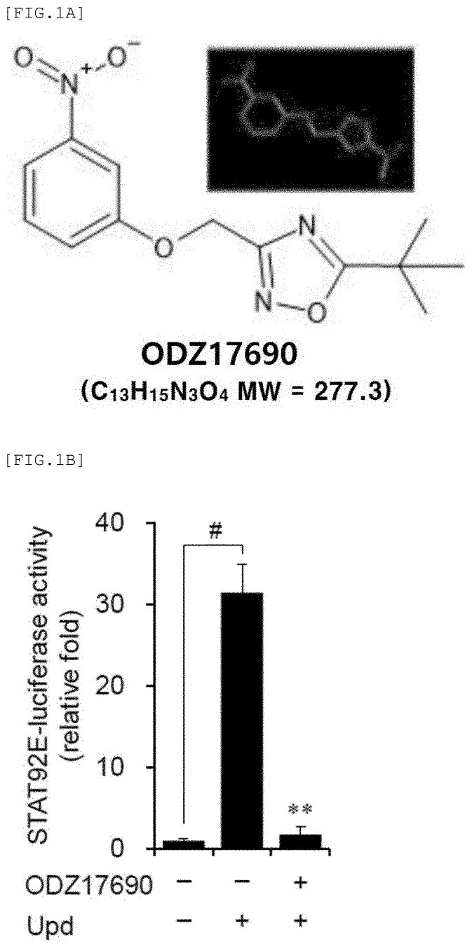

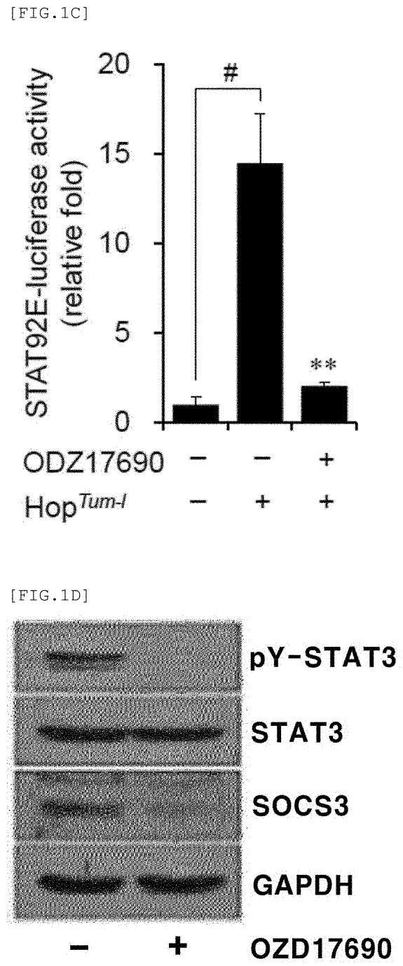

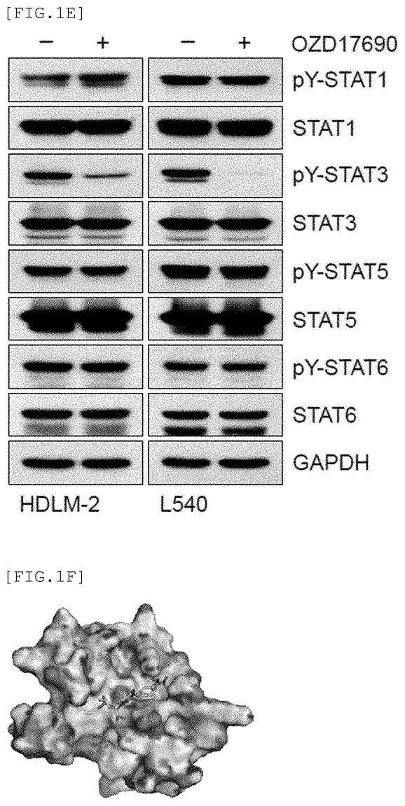

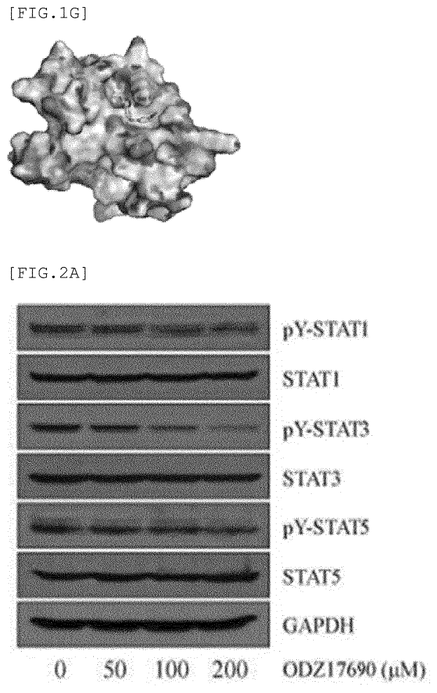

ODZ17690 (5-tert-butyl-3-(3-nitro-phenoxymethyl)-[1,2,4]oxadiazole) that was a compound having 3-phenoxymethyl-1,2,4-oxadiazole as a skeletal structure was identified using Drosophila cells, a human Hodgkin lymphoma cancer cell line (L540), and molecular models (FIG. 1A). First, in the luciferase assay using Drosophila cells, ODZ17690 effectively inhibited the expression of STAT92E activated by the activity of the ligand upd or Hop (equivalent to JAK in mammals) that was an upper level of STAT92E (corresponding to mammalian STAT) (FIGS. 1B and 1C). In L540 cells of human cancer cells as well, ODZ17690 effectively inhibited the phosphorylation of tyrosine residue 705 of STAT3 and the expression of SOCS3 of a lower regulator (FIG. 1D). In HDLM-2 cells of human cancer cells as well, ODZ17690 selectively inhibited phosphorylation of tyrosine residue 705 of STAT3 (FIG. 1E). In the binding-molecular model for the SH2 domain that was the binding site of STAT3, it was confirmed that the pTyr-Leu substrates of NSC-628869 and NSC-74859, which were selective STAT3 inhibitors and already known as standards, were selectively inhibited the SH2 domain and thus it was confirmed that the binding-molecular model fit well (FIG. 1F). ODZ17690 also bound well to the SH2 domain, had the lowest binding energy, and was similar to the control material (FIG. 1G).

### Confirmation of Selective STAT3 Inhibitory Action of ODZ17690 in Human Cancer Cell Line

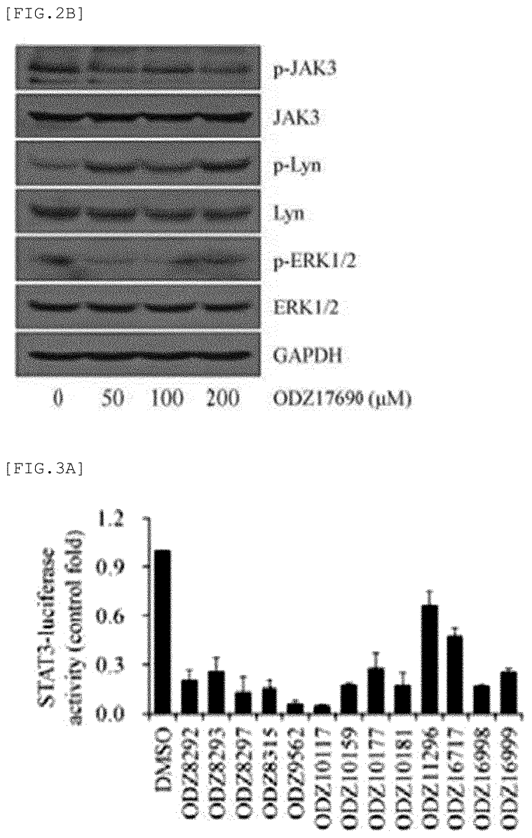

The selective STAT3 activity inhibitory action of ODZ17690 in human cancer cell lines was confirmed using a Hodgkin lymphoma cancer cell line L540. ODZ17690 exerted a stronger selective inhibitory action on STAT3 than on STAT1 or STAT5 (FIG. 2A). In addition, ODZ17690 exerted a weak inhibitory action on JAK3 in the upper signaling process but did not exert an inhibitory action on the Src family kinase Lyn or ERK signaling process (FIG. 2B). From the above results, it was confirmed that the selective STAT3 inhibitory action of ODZ17690 confirmed in the binding-molecular model and Drosophila cells was exerted in human cancer cells as well.

### Confirmation of Derivative Exerting Superior Efficacy Than ODZ17690

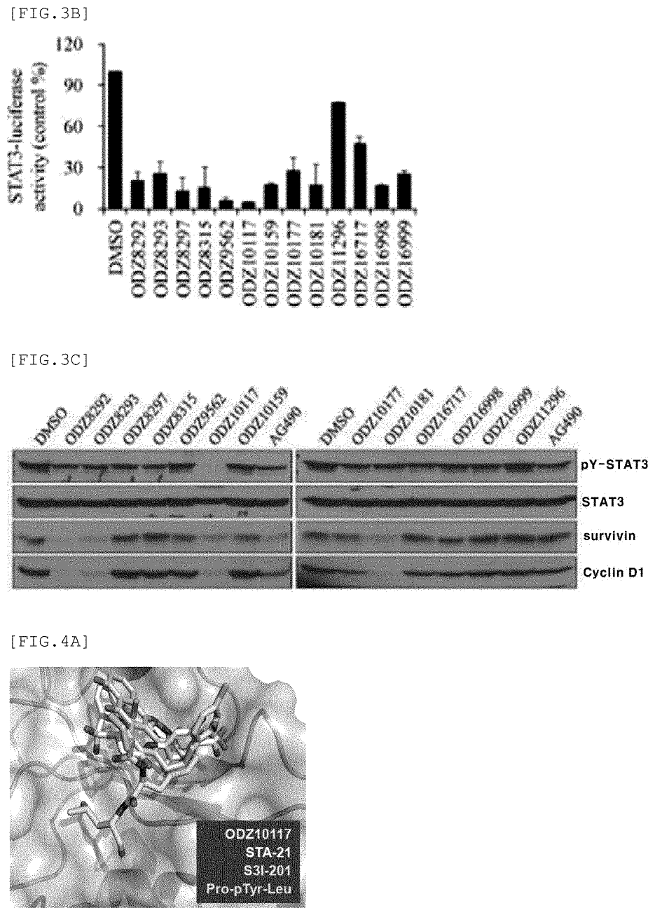

In order to identify compounds having superior selective STAT3 inhibitory efficacy than ODZ17690, luciferase assay was performed in Drosophila cells and human malignant breast cancer cell-derived MDA-MB-231 cells, which were constructed to selectively express STAT3 by constructing a STAT3-luciferase construct using 144 compounds having 3-phenoxymethyl-1,2,4-oxadiazole or 3-phenoxymethyl-1,2,4-thiadiazole as a skeletal structure. As a result, 13 compounds judged to exert excellent efficacy were selected as final candidates (FIGS. 3A and 3B). Moreover, Western blot analysis was performed using the 13 compounds to confirm again the compounds which were judged to exert excellent STAT3 inhibitory action, and ODZ10117 was judged to be extremely superior among these compounds (FIG. 3C).

### Confirmation of STAT3 Activity Inhibitory Action of ODZ10117 in Structure-Binding Molecular Model and Human Cancer Cell

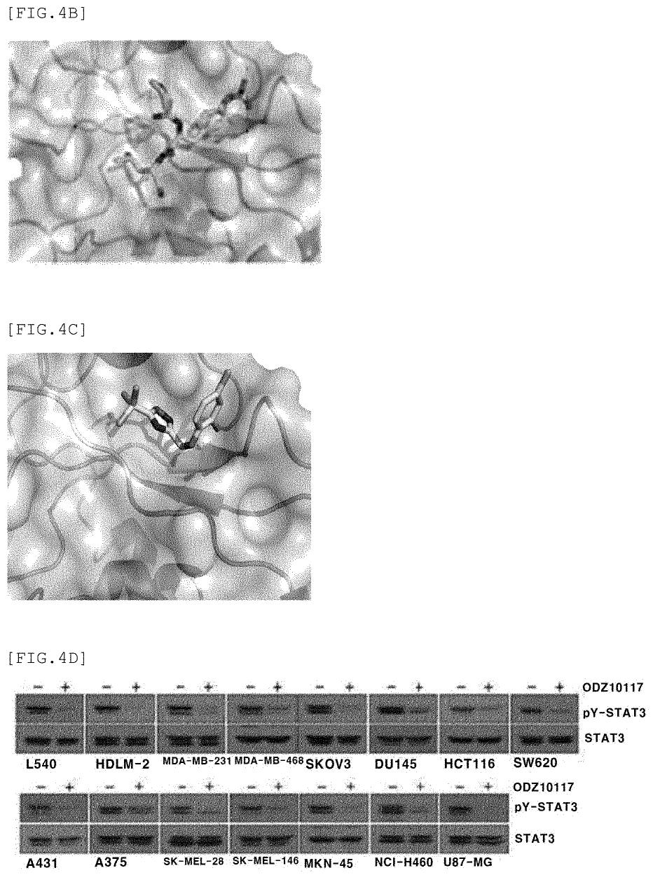

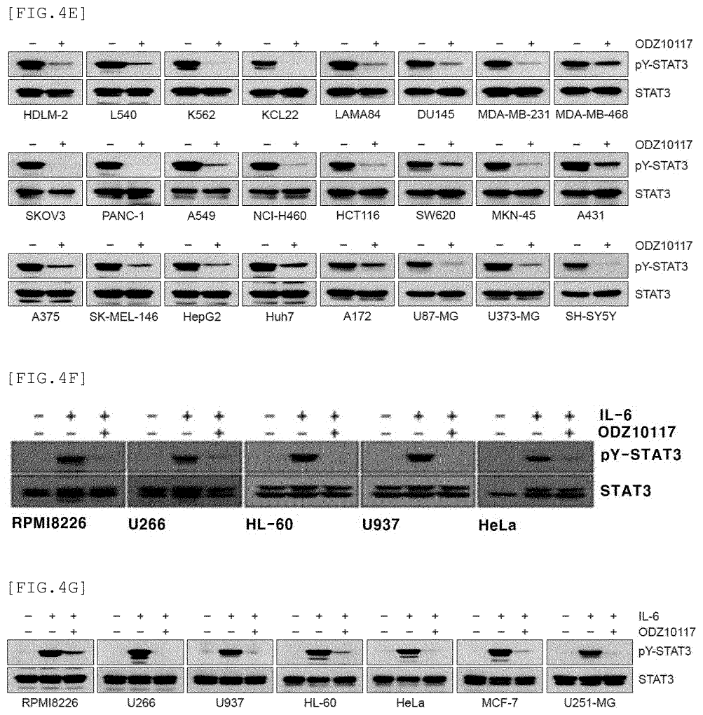

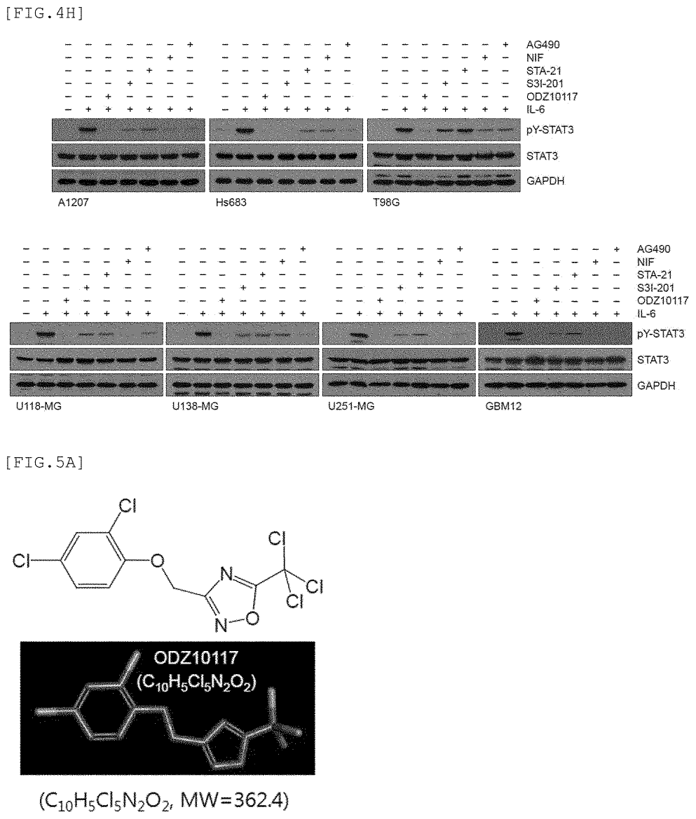

The selective STAT3 inhibitory efficacy of compound ODZ10117 was confirmed by a structure-binding molecular model. When the binding of the phosphorylated tyrosine residue to the SH2 domain was modeled, it was confirmed that ODZ10117 properly bound to the SH2 domain (turquoise blue). The binding of ODZ10117 to the SH2 domain was similar to those of NSC628869 (yellow) and NSC74859 (pink) known as selective STAT3 inhibitors, the free binding energy was also −11.14 kcal/mol for ODZ10117, −10.89 kcal/mol for NSC628869, and −10.01 kcal/mol for NSC74859, respectively so that ODZ10117 had lower binding energy, and it was confirmed that ODZ10117 had superior binding ability than the control materials (FIGS. 4A and 4C are for ODZ10117). In the X-ray complex structure complex, the binding of Pro-pTyr-Leu to the SH2 domain was indicated in green (FIGS. 4A and 4B).

The STAT3 activity inhibitory action of ODZ10117 was confirmed in various kinds of human cancer cell lines in which STAT3 was excessively activated. As a result, ODZ10117 inhibited the activity of STAT3 in all kinds of cell lines used in the experiment (FIGS. 4D and 4E). In addition, ODZ10117 also efficiently inhibited the activity of STAT3 induced by IL-6 (FIGS. 4F and 4G). Moreover, ODZ10117 more efficiently inhibited the activity of STAT3 induced by IL-6 than NSC628869 and NSC74859 known as selective STAT3 inhibitors (FIG. 4H). Through the above results, it was confirmed that ODZ10117 exerted excellent inhibitory action on STAT3 activated in human cancer cell lines.

### Confirmation of Selective STAT3 Activity Inhibitory Action of ODZ10117 in Human Cancer Cell Line

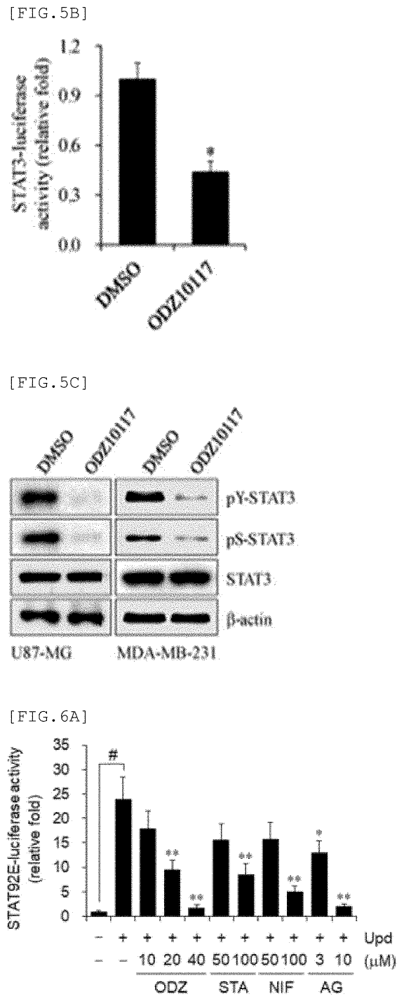

The selective STAT3 activity inhibitory action of the compound ODZ10117 was confirmed in human malignant glioblastoma and human malignant breast cancer cell lines U87-MG and MDA-MB-231 cells. The compound ODZ10117 is a compound having a structure of 3-(2,4-dichloro-phenoxymethyl)-5-trichloromethyl-[1,2,4]oxadiazole (FIG. 5A). First, in order to confirm the promoter activity of STAT3, the pSTAT3-TA-luciferase DNA construct was transformed into a HEK293T cell line, and the efficacy of ODZ10117 was confirmed. As a result, ODZ10117 significantly decreased the promoter activity of STAT3 as compared with the DMSO-treated group (FIG. 5B).

Next, Western blot was performed in order to analyze the degree of activity on tyrosine and serine residues of STAT3 protein. As a result, ODZ10117 significantly inhibited phosphorylation of tyrosine 705 and serine 727 residues of STAT3 in U87-MG and MDA-MB-231 cells (FIG. 5C).

### Confirmation of Selective STAT92E Activity Inhibitory Action of ODZ10117 in Drosophila Cell Line

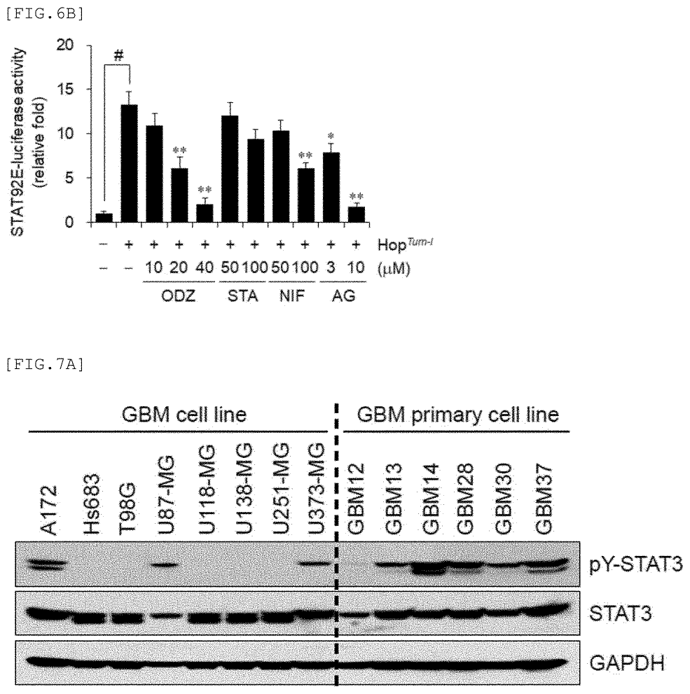

The selective STAT92E (corresponding to mammalian STAT) activity inhibitory action of ODZ10117 was confirmed in Drosophila cells. As a result, ODZ10117 significantly decreased the promoter activity of STAT92E as compared with the DMSO-treated group in a concentration-dependent manner (FIGS. 6A and 6B).

### Confirmation of Selective STAT92E Activity Inhibitory Action of ODZ10117 in Human Malignant Glioblastoma

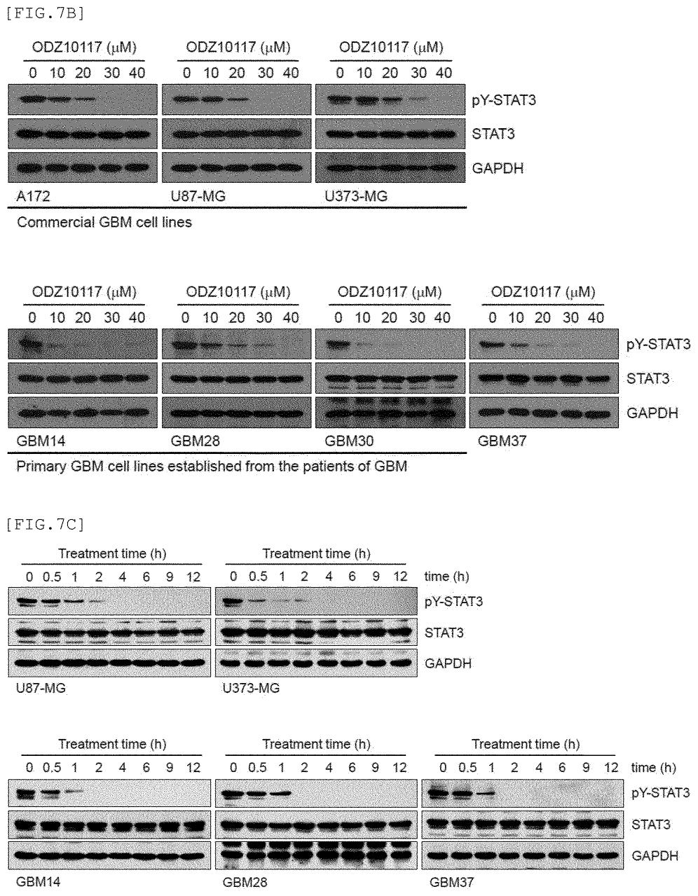

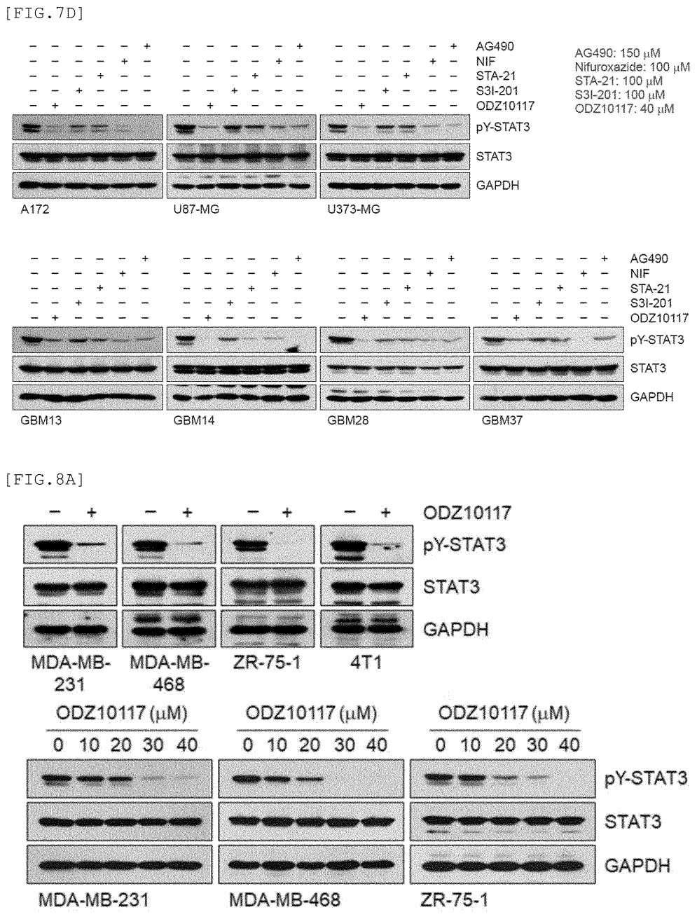

The selective STAT3 activity inhibitory action of ODZ10117 was confirmed in human malignant glioblastoma. First, Western blot analysis was performed using commercially available human malignant glioblastoma and human malignant glioblastoma primary culture cell lines to select STAT3-phosphorylated human malignant glioblastoma (FIG. 7A). Next, Western blot was performed to analyze the degree of activity on tyrosine of STAT3 protein in STAT3-phosphorylated human malignant glioblastoma. As a result, ODZ10117 inhibited the phosphorylation of tyrosine 705 of STAT3 in STAT3-phosphorylated human malignant glioblastoma in a concentration-dependent manner (FIG. 7B), and phosphorylation of STAT3 was completely inhibited within 2 hours after ODZ10117 treatment (FIG. 7C). In addition, ODZ10117 exerted superior STAT3 inhibitory efficacy to NSC628869 and NSC74859 known as selective STAT3 inhibitors (FIG. 7D).

### Confirmation of Selective STAT92E Activity Inhibitory Action of ODZ10117 in Human Malignant Breast Cancer Cell Line

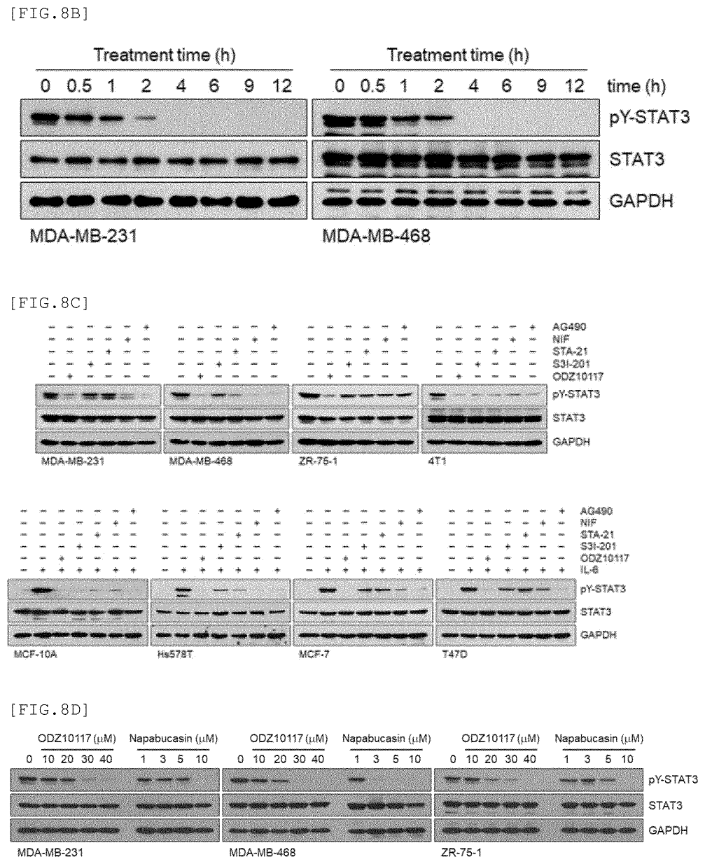

The selective STAT3 activity inhibitory action of ODZ10117 was confirmed in human malignant breast cancer cell lines. Western blot was performed to analyze the degree of activity on tyrosine of STAT3 protein in human malignant breast cancer cell lines. As a result, ODZ10117 inhibited the phosphorylation of tyrosine 705 of STAT3 in human malignant breast cancer cell lines in a concentration-dependent manner (FIG. 8A), and phosphorylation of STAT3 was completely inhibited within 2 hours after ODZ10117 treatment (FIG. 8B). In addition, ODZ10117 exerted superior STAT3 inhibitory efficacy to NSC628869 and NSC74859 known as selective STAT3 inhibitors (FIG. 8C). Next, the selective STAT3 activity inhibitory actions of ODZ10117 and napabucasin known as an anticancer substance in human malignant breast cancer cell lines were compared with each other. Western blot was performed to analyze the degree of activity on tyrosine of STAT3 protein in human malignant breast cancer cell lines. As a result, ODZ10117 exerted STAT3 inhibitory efficacy similar to that of napabucasin at high concentrations (FIG. 8D).

### Confirmation of STAT3 Dimerization, Nuclear Translocation and Transcriptional Activity Inhibitory Action of ODZ10117

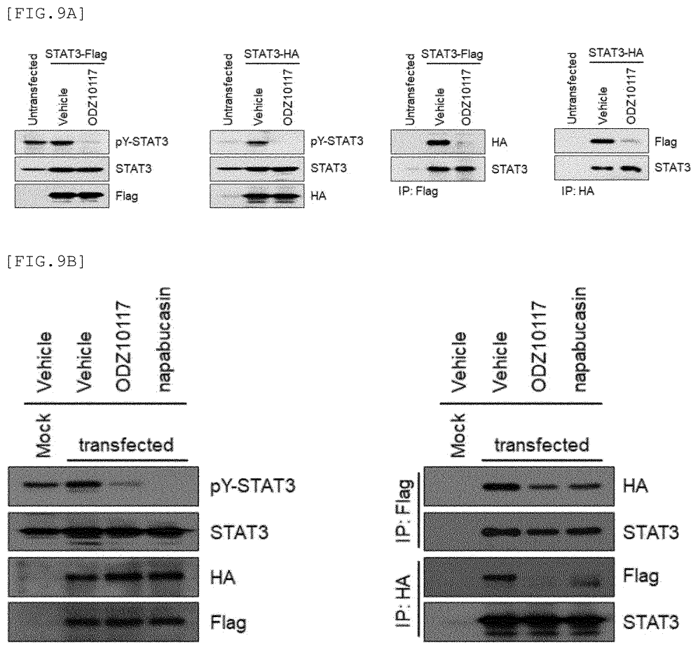

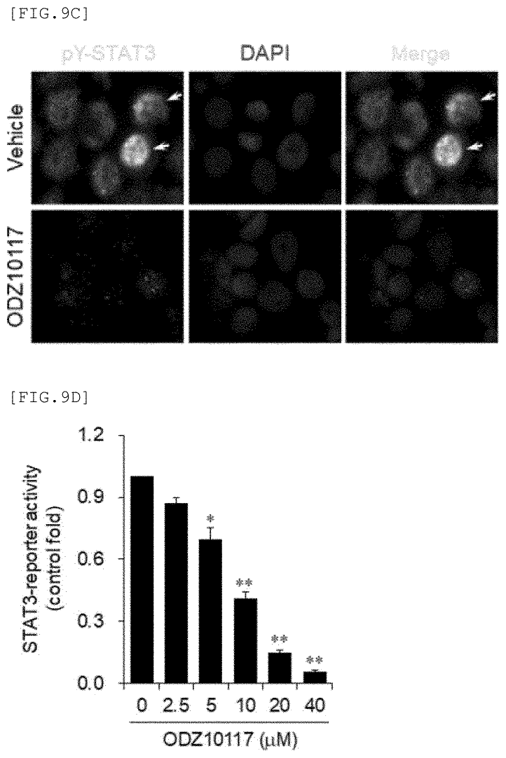

The STAT3 dimerization, nuclear translocation and transcriptional activity inhibitory action of the compound ODZ10117 was confirmed. First, in order to confirm the STAT3 dimerization, the STAT3-Flag DNA construct or STAT3-HA DNA construct was transformed into a HEK293T cell line, and immunoprecipitation and western blot analysis were performed. As a result, ODZ10117 inhibited STAT3 dimerization (FIG. 8A). In addition, ODZ10117 exerted superior STAT3 dimerization inhibitory efficacy to napabucasin (FIG. 9B). Next, in order to confirm the nuclear translocation of STAT3, the nuclear translocation of STAT3 was observed under a fluorescence microscope using the cell lines. As a result, ODZ10117 further inhibited nuclear translocation of STAT3 as compared with the control group (FIG. 9C). Next, in order to confirm the transcriptional activity of STAT3, plasmid DNA having STAT3-TA-luciferase as a reporter construct was transformed into a HEK293T cell line, the HEK293T cell line was treated with ODZ10117 at various concentrations, and then luciferase assay was performed. As a result, ODZ10117 inhibited the transcriptional activity of STAT3 in a concentration-dependent manner (FIG. 9D).

### Confirmation of Cell Survival Inhibitory Action of ODZ10117 in STAT3-Activated Human Cancer Cell Line

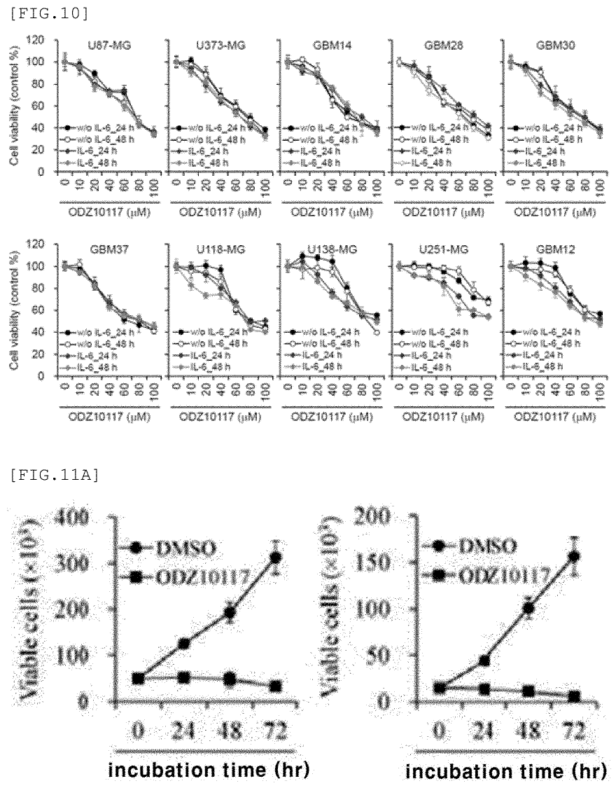

STAT3 activated in cancer cells increases cell proliferation and growth by increasing the expression of proteins associated with cell survival. Hence, when human malignant glioblastoma which was treated with IL-6 for 24 or 48 hours or was not treated with IL-6 was treated with ODZ10117 at various concentrations and the cell viability was confirmed, the cell survival was inhibited in the ODZ10117-treated groups as compared with the ODZ10117-untreated group in a concentration-dependent manner (FIG. 10).

### Confirmation of Cancer Cell Apoptosis Inducing Action of ODZ10117

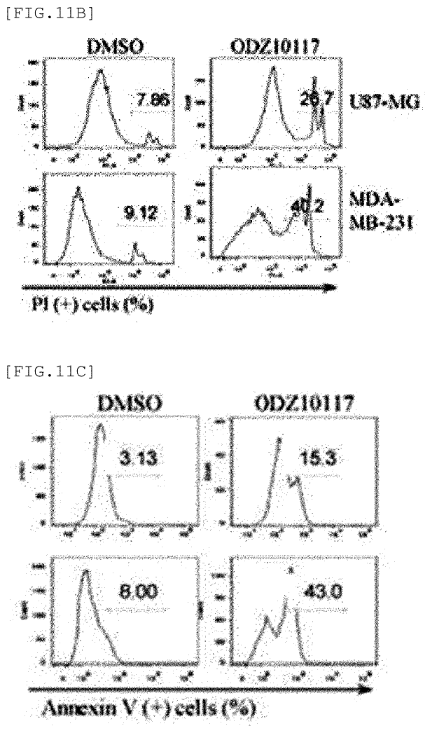

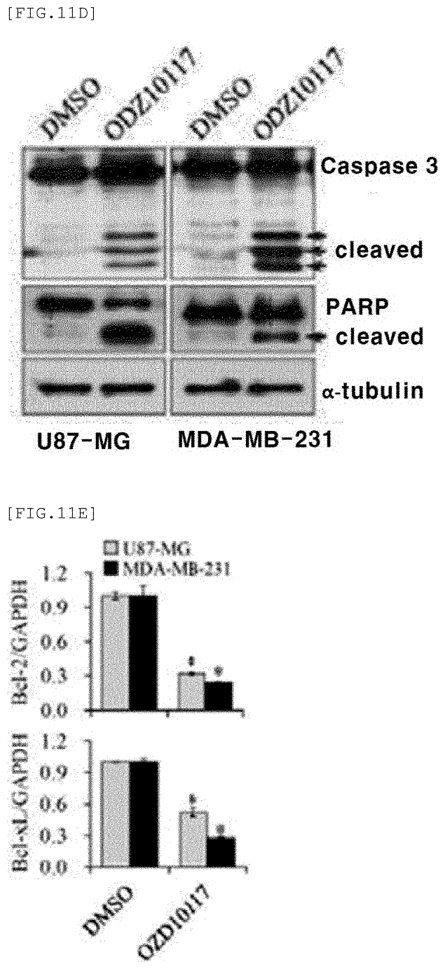

STAT3 activated in cancer cells increases cell proliferation and growth by increasing the expression of proteins associated with cell survival and cell cycle. Hence, when the effect of ODZ10117 on cell proliferation of U87-MG and MDA-MB-231 cell lines was confirmed by time period, ODZ10117 effectively inhibited the proliferation of the two cell lines as compared with the DMSO-treated group as a control group, (FIG. 11A). Next, in order to analyze the apoptosis effect of ODZ10117 on the two cell lines, the two cell lines were treated with the compound ODZ10117 for 48 hours and stained with PI (propidium iodide) and Annexin V, and flow cytometry was performed. As a result, ODZ10117 induced apoptosis of about 15.3% to 26.7% for the U87-MG cell line and apoptosis of about 40.2% to 43.0% for the MDA-MB-231 cell line (FIGS. 11B and 11C). Since apoptosis involves expression of various genes, the degrees of activity of caspase-3 and PARP, which were representative pro-apoptotic genes involved in apoptosis, were analyzed through Western blot analysis. It was confirmed that ODZ10117 significantly increased the amount of fragmented caspase-3 and PARP proteins and the activity of these proteins increased. Through these results, it was confirmed that ODZ10117 induced apoptosis by increasing the activity of caspase-3 and PARP (FIG. 11D). Next, the expression levels of the anti-apoptotic genes Bcl-2 and Bcl-XL were analyzed by real-time polymerase chain reaction. As a result, ODZ10117 effectively inhibited the expression of these genes (FIG. 11E). Through the above results, it was confirmed that ODZ10117 increased the apoptosis of cancer cells by increasing the activity of the pro-apoptotic genes but decreasing the expression of the anti-apoptotic genes.

### Confirmation of Cell Migration and Invasion Inhibitory Action of ODZ10117

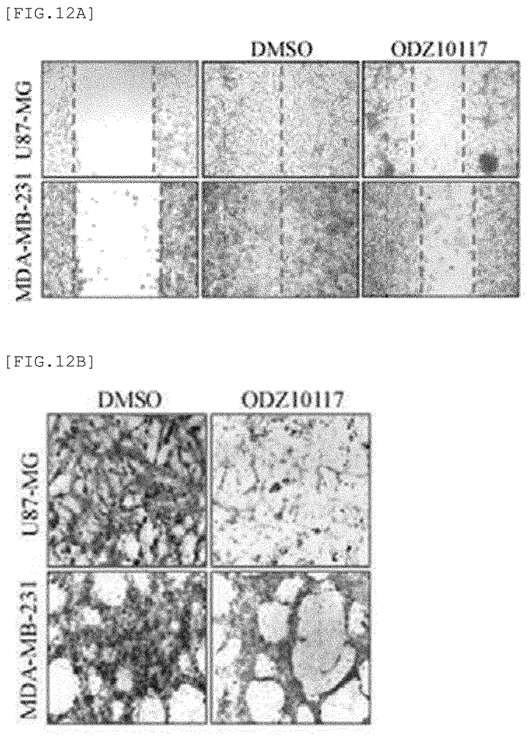

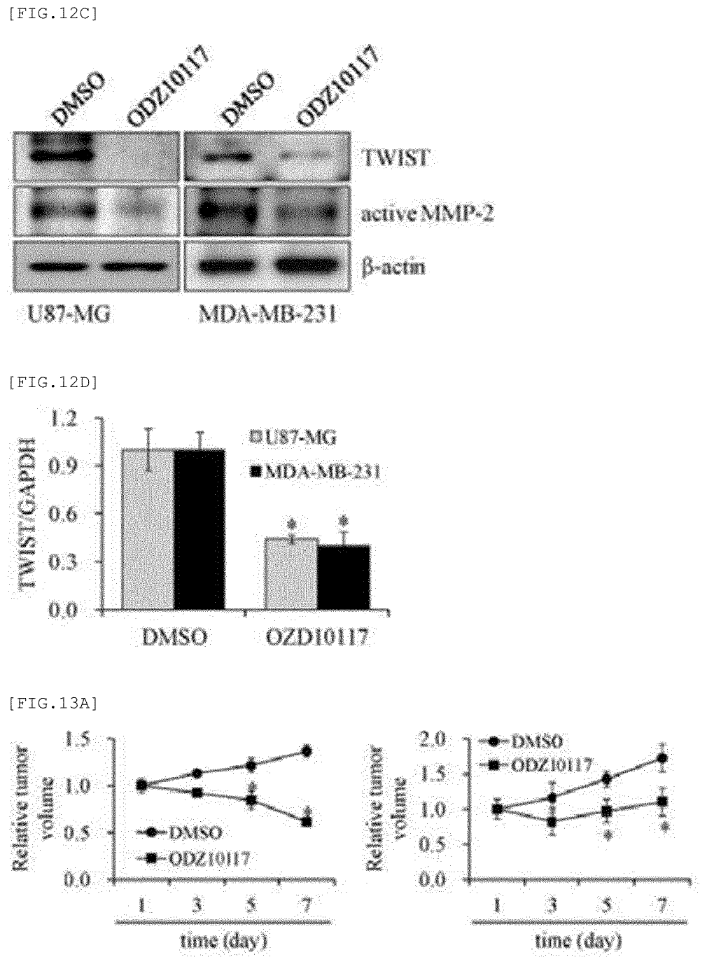

The efficacy of ODZ10117 on cell mobility and invasiveness associated with metastasis of human tumor cells was confirmed. First, wound healing assay was performed to confirm the effect of ODZ10117 on cell mobility. When U87-MG and MDA-MB-231 cells were treated with the compound ODZ10117 for 24 hours and cell migration was observed under a microscope, cell migration was active in the control group treated with DMSO but ODZ10117 significantly inhibited the migration of these cells (FIG. 12A).

In order to verify the effect of ODZ10117 on the cell invasion of cancer cells, cell invasion assay was performed. U87-MG and MDA-MB-231 cells were dispensed into Matrigel, and the lower chamber was filled with a culture solution containing fibronectin. The U87-MG and MDA-MB-231 cells were treated with ODZ10117 and cultured for 24 hours, and then the degree of cell invasion into the lower chamber was observed under a microscope. As a result, ODZ10117 significantly decreased cell invasion in both the two cell lines as compared with the control group treated with DMSO (FIG. 12B).

Various factors are involved in cancer cell migration and invasion. Accordingly, the efficacy of ODZ10117 on the expression of Twist and activity of MMP-2, which were one of the representative factors involved in cell migration and invasion, was confirmed. As a result, when U87-MG and MDA-MB-231 cells were treated with ODZ10117 for 24 hours, the expression of Twist and the activity of MMP-2 in U87-MG and MDA-MB-231 cells significantly decreased (FIGS. 12C and 12D).

### Confirmation of Tumor Growth Inhibitory Effect of ODZ10117 in Human Tumor Cell Xenograft

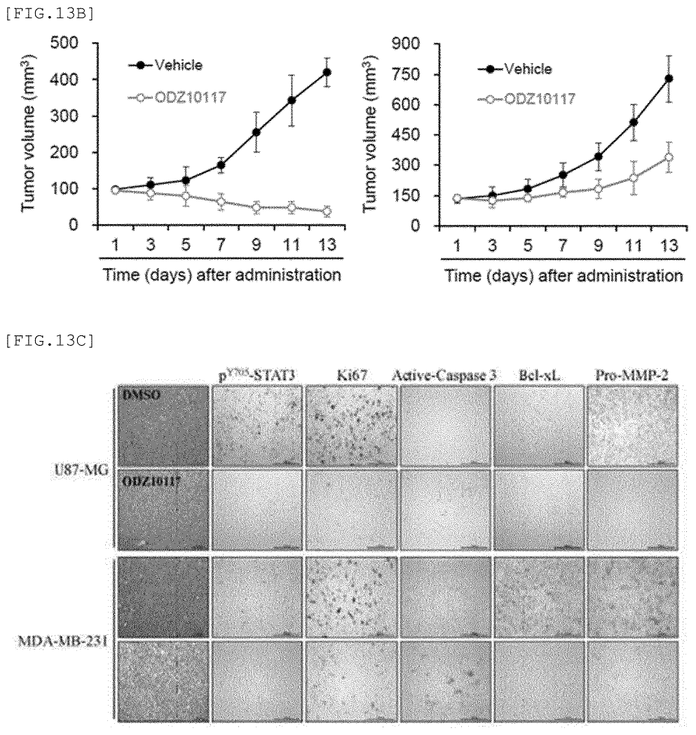

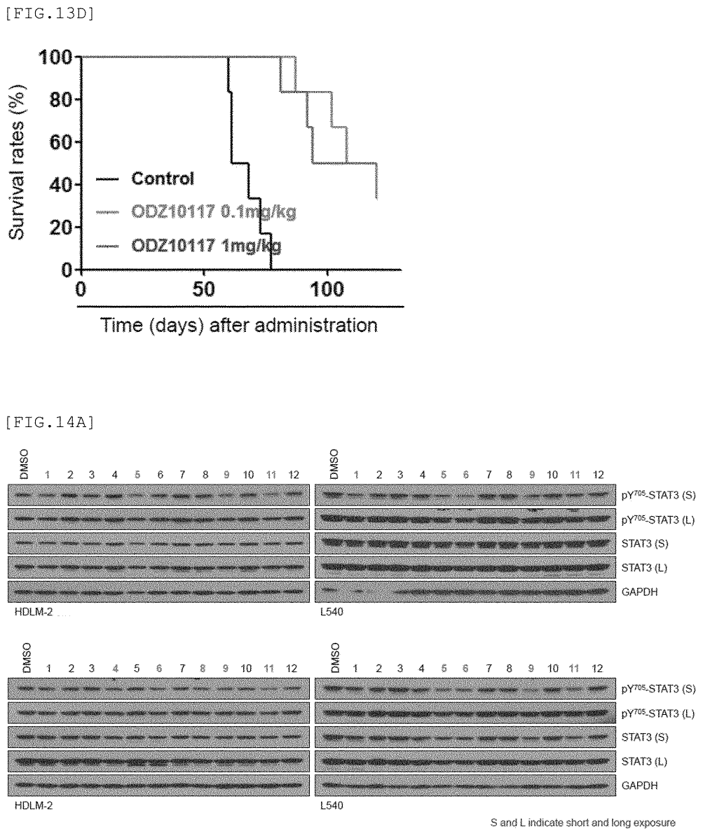

In order to confirm the efficacy of ODZ10117 on the STAT3 activity inhibition through animal experiments, U87-MG and MDA-MB-231 cells of human cancer cell lines were respectively mixed with 25% Matrigel and injected into the back of immunodeficient mice (BALB/c nude mice), and the mice whose tumors grew to an appropriate size were selected after 4 weeks. DMSO or vehicle and ODZ10117 were injected directly and periodically into the tumor masses of these mice on days 0, 3, 5, 7, 9, 11, and 13, and the tumor growth was measured. As a result, ODZ10117 significantly inhibited tumor growth as compared with the control group to which DMSO was injected (FIGS. 13A and 13B).

On the 7th day of drug administration, the mice were anesthetized and sacrificed to remove the tumor masses. The tumor tissues were fixed with formalin, embedded in paraffin, and cut to obtain tissue sections. The phosphorylation of tyrosine 705 residue of STAT3, expression of Ki67, activity of caspase-3, and expression of Bcl-XL and pro/activated MMP2 in the tissue sections were analyzed. As a result, phosphorylation of STAT3 significantly decreased in the tumor tissues to which ODZ10117 was injected as compared with the control group to which DMSO was injected. In addition, ODZ10117 significantly decreased the expression of Ki67 that was one of the cell growth factors, Bcl-XL associated with apoptosis resistance, and pro/active MMP2 associated with cell invasion. Moreover, ODZ10117 increased the activity of caspase-3 associated with apoptosis (FIG. 13C).

In addition, DMSO or ODZ10117 was directly injected into mice to which U87-MG cells of a human cancer cell line was injected, and the survival rate was confirmed. As a result, the survival time increased in the mice to which ODZ10117 was injected as compared with the control group to which DMSO was injected (FIG. 13D).

These results suggest that ODZ10117 selectively inhibits the activity of STAT3 and thus inhibits the growth, proliferation, invasion and metastasis of tumor cells, induces the apoptosis of cancer cells, and can inhibit the growth of tumors.

### Screening of Selective STAT3 Inhibitory Compounds Through Structure-Based Virtual Screening

Through structure-based screening, 46 compounds expected to selectively inhibit STAT3 were obtained. Information on the 46 compounds is presented in the following Table 1.

TABLE 1

| | | Molecular | |
|---|---|---|---|
| o | ID | weight | Mol Name |
| 1 | 4009697 | 280.2 | 2-(2-aminoethyl)-4(3H)-quinazolinone |
| | | | dihydrochloride hydrate |
| 2 | 7952466 | 453.9 | 2-chloro-5-{5-[(2-{[(4- |
| | | | methylphenyl)sulfonyl]amino]ethyl)thio]-lH- |
| | | | tetrazol-l-yl]benzoic acid |
| 3 | 5118596 | 293.3 | l-(3-pyridinyl)-2,3,4,9-tetrahydro-lH-beta- |
| | | | carboline-3-carboxylic acid |
| 4 | 9034114 | 301.4 | N-[2-(lH-benzimidazol-2- |
| | | | yl)ethyl]benzenesulfonamide |
| 5 | 5277898 | 207.2 | 5-(2-hydroxybenzylidene)-l,3-thiazolidin-4-one |
| 6 | 9003625 | 287.3 | N-(1H-benzimidazol-2-ylmethyl)benzenesulfonamide |
| 7 | 9199753 | 282.3 | N-(methylsulfonyl)tryptophan |
| 8 | 5131328 | 308.3 | l-(4-hydroxyphenyl)-2,3,4,9-tetrahydro-lH-beta- |
| | | | carboline-3-carboxylic acid |
| 9 | 5154969 | 431.9 | 2-chloro-N-({[4-hydroxy-6-(2-phenylvinyl)-l,3,5- |
| | | | triazin-2-yl]amino]carbonyl)benzenesulfonamide |
| 10 | 7815802 | 342.8 | N~4~-(3-chlorophenyl)-N~2~-(3- |
| | | | isopropoxypropyl)asparagine |
| 11 | 7579859 | 345.3 | 3-({[3- |
| | | | (trifluoromethyl)phenyl]sulfonyl}amino)benzoic |
| | | | acid |
| 12 | 5107319 | 283.5 | 3-(2-aminoethyl)-5-(aminosulfonyl)-lH-indole-2- |
| | | | carboxylic acid |
| 13 | 9235510 | 297.4 | N-[5-(4-pyridinylmethyl)-1,4,5,6-tetrahydro- |
| | | | 1,3,5-triazin-2-yl]-2-propanesulfonamide |
| 14 | 5175489 | 321.8 | N-(1H-benzimidazol-2-ylmethyl)-4- |
| | | | chlorobenzenesulfonamide |
| 15 | 7841794 | 229.3 | 2-(l-azepanylmethyl)-lH-benzimidazole |
| 16 | 7994850 | 293.3 | 8-[(2,6-dimethyl-4-morpholinyl)methyl]-3-methyl- |
| | | | 3,7-dihydro-lH-purine-2,6-dione |
| 17 | 4010128 | 205.2 | 2-(2-aminoethyl)-2,3-dihydrophthalazine-l,4-dione |
| 18 | 9326315 | 265.4 | (1H-benzimidazol-2-ylmethyl)(2-phenylpropyl)amine |
| 19 | 7973191 | 425.5 | N-{3-[5-(3-fluorophenyl)-l-(methylsulfonyl)-4,5- |
| | | | dihydro-lH-pyrazol-3-yl]phenyl}ethanesulfonamide |
| 20 | 5175507 | 356.2 | N-(lH-benzimidazol-2-ylmethyl)-3,4- |
| | | | dichlorobenzenesulfonamide |
| 21 | 9320045 | 315.3 | N-{4-[(2-oxo-l-imidazolidinyl)carbonyl]phenyl}-2- |
| | | | thiophenecarboxamide |
| 22 | 9309428 | 309.3 | N-{4-[(2-oxo-l- |
| | | | imidazolidinyl)carbonyl]phenyl}benzamide |
| 23 | 9059590 | 374.8 | 5-chloro-N-cyclopropyl-2-[2-(4-morpholinyl)-2- |
| | | | oxoethoxy]benzenesulfonamide |
| 24 | 9134776 | 298.3 | 4-{[(4-hydroxyphenyl)amino]sulfonyl}-2- |
| | | | thiophenecarboxamide |
| 25 | 7356076 | 343.8 | 2-amino-N-(4-chloro-2,5-dimethoxyphenyl)-4-oxo- |
| | | | 5,6-dihydro-4H-l,3-thiazine-6-carboxamide |
| 26 | 9127934 | 338.8 | N-(3-chloro-2-methylphenyl)-3- |
| | | | [(methylsulfonyl)amino]benzamide |
| 27 | 7400955 | 230.3 | 4-cinnamoyl-2-piperazinone |
| 28 | 9120911 | 304.4 | N-(2-methylphenyl)-3- |
| | | | ((methylsulfonyl)amino]benzamide |
| 29 | 7802614 | 363.4 | N-benzyl-3-({[(4- |
| | | | fluorophenyl)amino]carbonyl}amino)benzamide |
| 30 | 9287619 | 311.4 | 3-[(anilinocarbonyl)amino]-N-(tert- |
| | | | butyl)benzamide |
| 31 | 5360857 | 315.4 | N-[2-(1H-benzimidazol-2-yl)ethyl]-4- |
| | | | methylbenzenesulfonamide |
| 32 | 5175505 | 301.4 | N-(1H-benzimidazol-2-ylmethyl)-4- |
| | | | methylbenzenesulfonamide |
| 33 | 9154810 | 305.4 | 3-[(methylsulfonyl)amino]-N-(4- |
| | | | pyridinylmethyl)benzamide |
| 34 | 9003359 | 364.2 | 2-{[(6-bromo-l,3-benzodioxol-5-yl)methyl]thio}- |
| | | | 3H-imidazo[4,5-b]pyridine |
| 35 | 7806202 | 339.3 | l-[(2,3-dioxo-l,2,3,4-tetrahydro-6- |
| | | | quinoxalinyl)sulfonyl]proline |
| 36 | 9015042 | 307.4 | 5-ethyl-N-(3-hydroxyphenyl)-2- |
| | | | methoxybenzenesulfonamide |
| 37 | 9203703 | 310.4 | 3-({4-[(3-hydroxypropyl)amino]-2- |
| | | | quinazolinyl}amino)phenol |
| 38 | 5186975 | 618.7 | N,N′-[methylenebis(2-hydroxy-4,1- |
| | | | phenylene)]bis(2,2-diphenylacetamide) |
| 39 | 9272567 | 324.4 | N-[5-(tetrahydro-2-furanylmethyl)-l,4,5,6- |
| | | | tetrahydro-l,3,5-triazin-2-yl]benzenesulfonamide |
| 40 | 9224447 | 236.3 | N-[5-(3-hydroxypropyl)-l,4,5,6-tetrahydro-l,3,5- |
| | | | triazin-2-yl]methanesulfonamide |
| 41 | 9336135 | 339.3 | 2-methoxy-N-{4-[(2-oxo-l- |
| | | | imidazolidinyl)carbonyl]phenyl}benzamide |
| 42 | 6907387 | 258.3 | 5-(2,3-dimethyl-lH-indol-5-yl)-4-methyl-2,4- |
| | | | dihydro-3H-l,2,4-triazole-3-thione |
| 43 | 9134168 | 353.4 | 3-[(phenylsulfonyl)amino]-N-3-pyridinylbenzamide |
| 44 | 9143017 | 424.5 | 2-({3-[(methylsulfonyl)amino]benzoyl]amino)-N-(3- |
| | | | pyridinylmethyl)benzamide |
| 45 | 9107321 | 304.3 | 1-(4-hydroxyphenyl)-5-oxo-N-(tetrahydro-2- |
| | | | furanylmethyl)-3-pyrrolidinecarboxamide |
| 46 | 9157889 | 367.4 | 3-{[(4-methylphenyl)sulfonyl]amino}-N-3- |
| | | | pyridinylbenzamide |

### Confirmation of Selective STAT3 Activity Inhibitory Action of Selected Compound in Human Cancer Cell Line Through Structure-Based Virtual Screening

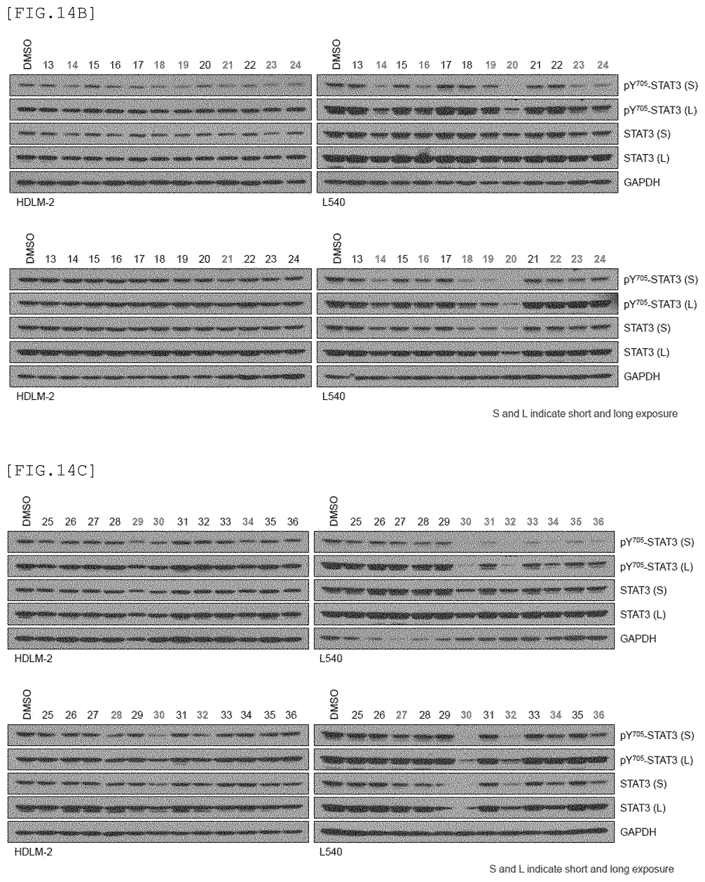

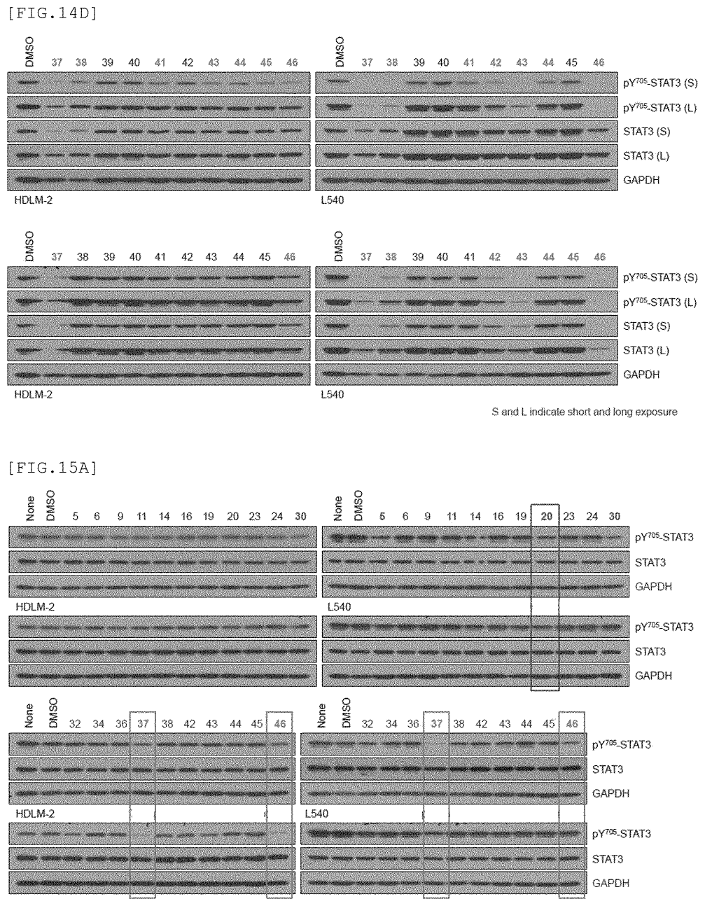

In order to find out whether the 46 compounds in Table 1 above exert selective STAT3 inhibitory efficacy, Western blot analysis was performed in human Hodgkin lymphoma cancer cell lines L540 and HDLM-2 using the 46 compounds. As a result, among the 46 compounds, 33 compounds (Compounds No. 1, 4, 5, 6, 8, 9, 11, 14, 16, 18, 19, 20, 21, 22, 23, 24, 27, 28, 29, 30, 31, 32, 33, 34 , 35, 36, 37, 38, 41, 42, 43, 44, 45, and 46 in Table 1 above) which were judged to exert superior STAT3 inhibitory efficacy were first screened (FIGS. 14A to 14D).

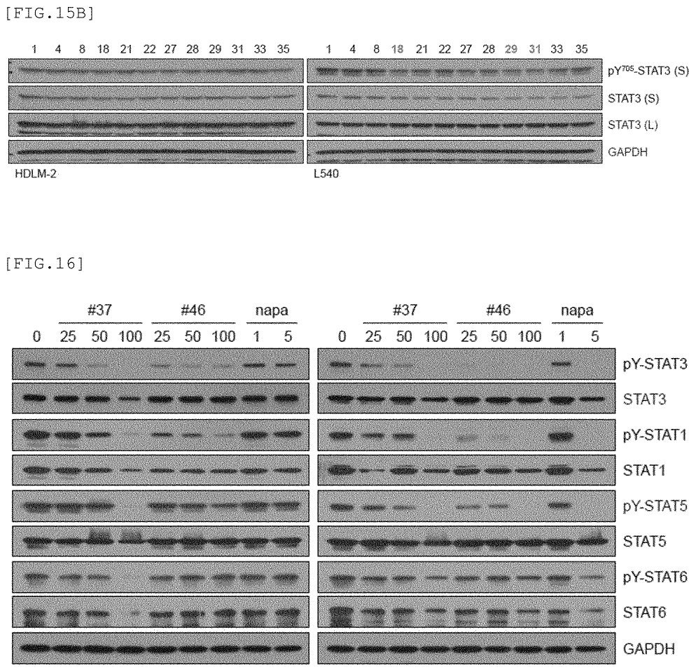

Next, among the 33 compounds which were first screened in human Hodgkin lymphoma cancer cell lines L540 and HDLM-2, the human Hodgkin lymphoma cancer cell lines L540 and HDLM-2 were treated with 50 μM of 21 compounds (Compounds No. 5, 6, 9, 11, 14, 16, 19, 20, 23, 24, 30, 32, 34, 36, 37, 38, 42, 43, 44, 45, 46 in Table 1 above) or 100 μM of 12 compounds (Compounds No. 1, 4, 8, 18, 21, 22, 27, 28, 29, 31, 33, 35, 41 in Table 1 above), and Western blot analysis was performed. As a result, among the 33 compounds first screened, 2 compounds (Compounds No. 37 and 46 in Table 1 above) which were judged to exert superior STAT3 inhibitory efficacy were secondarily screened (FIGS. 15A and 15B). Next, human Hodgkin lymphoma cancer cell lines L540 and HDLM-2 were treated with the two compounds (Compounds No. 37 and 46 in Table 1 above) secondarily selected at various concentrations, and Western blot analysis was performed. As a result, both the two compounds secondarily selected significantly inhibited the phosphorylation of STATdml tyrosine 705 in a concentration-dependent manner (FIG. 16).

The specific parts of the present invention have been described in detail above, and it is obvious for those skilled in the art that these specific techniques are only preferred embodiments and the scope of the present invention is not limited thereto. Accordingly, the substantial scope of the present invention will be defined by the appended claims and their equivalents.

### INDUSTRIAL APPLICABILITY

The present invention relates to a compound having STAT3 inhibitory activity, or a pharmaceutically acceptable salt, solvate or hydrate thereof, and pharmaceutical uses thereof, and the compound having STAT3 inhibitory activity of the present invention or a pharmaceutically acceptable salt, solvate or hydrate thereof can be usefully utilized for the prevention and treatment of various STAT3-related diseases associated with cancer, autoimmune diseases, inflammatory diseases, and the like.

1. A compound selected from the group consisting of compounds represented by the following Chemical Formulas 1 to 60, or a pharmaceutically acceptable salt, solvate or hydrate of the compound:

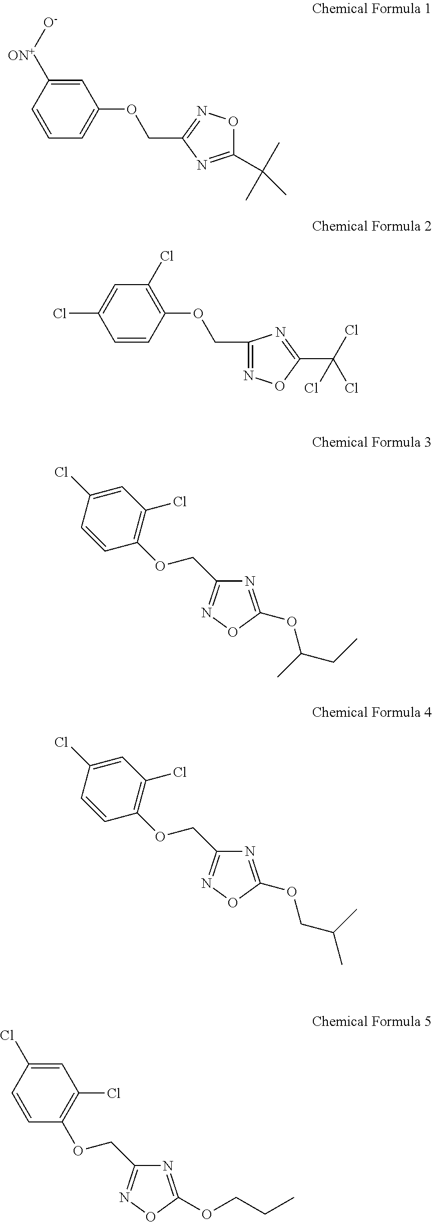

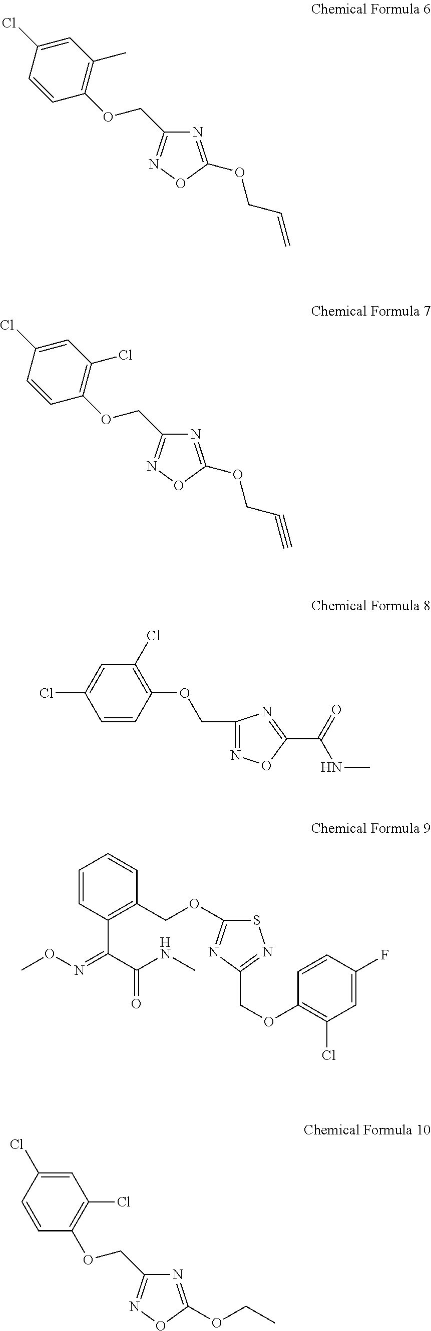

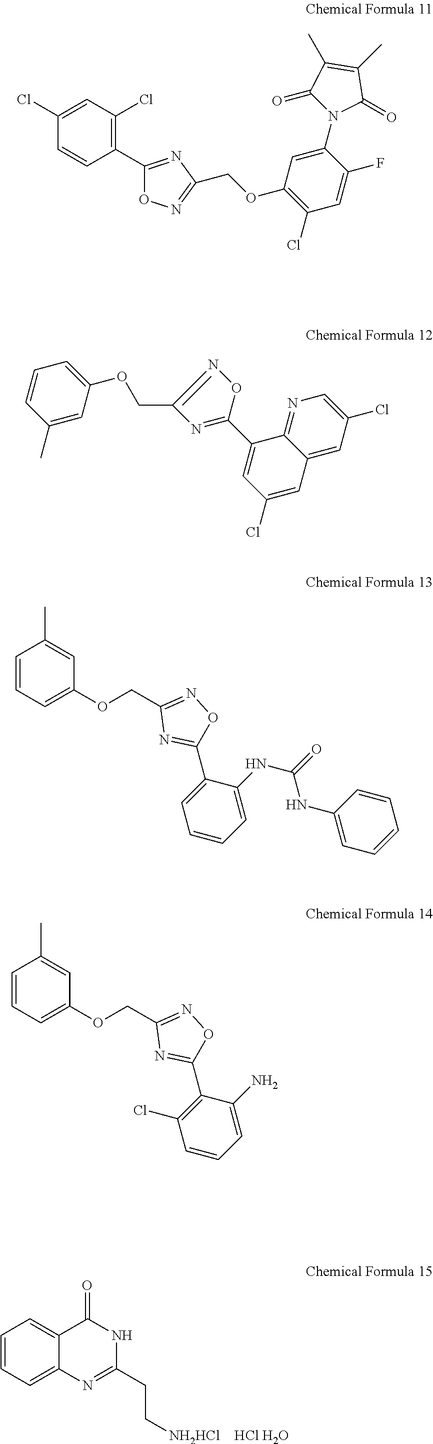

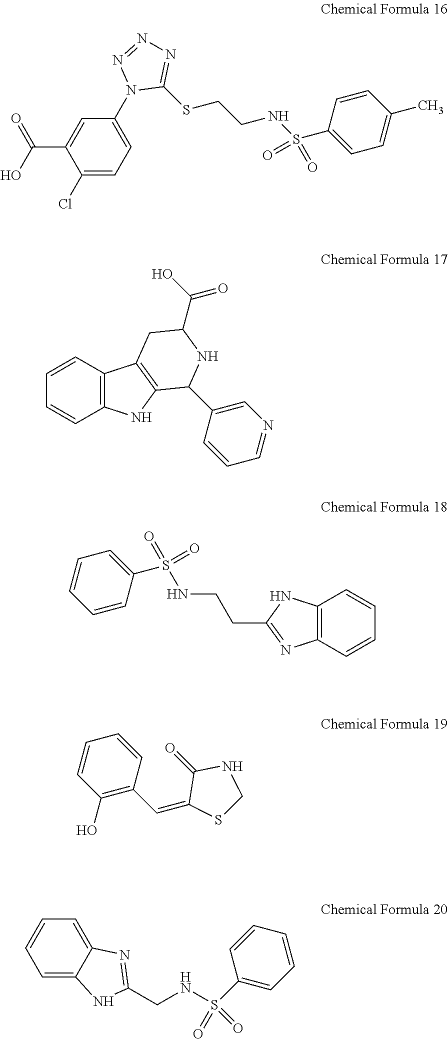 [SMILES: Cc1ccc(S(=O)(=O)NCCSc2nnnn2-c2ccc(Cl)c(C(=O)O)c2)cc1 ; O=C(O)C1Cc2c([nH]c3ccccc23)C(c2cccnc2)N1 ; O=S(=O)(NCCc1nc2ccccc2[nH]1)c1ccccc1 ; O=C1NCS/C1=C/c1ccccc1O ; O=S(=O)(NCc1nc2ccccc2[nH]1)c1ccccc1]

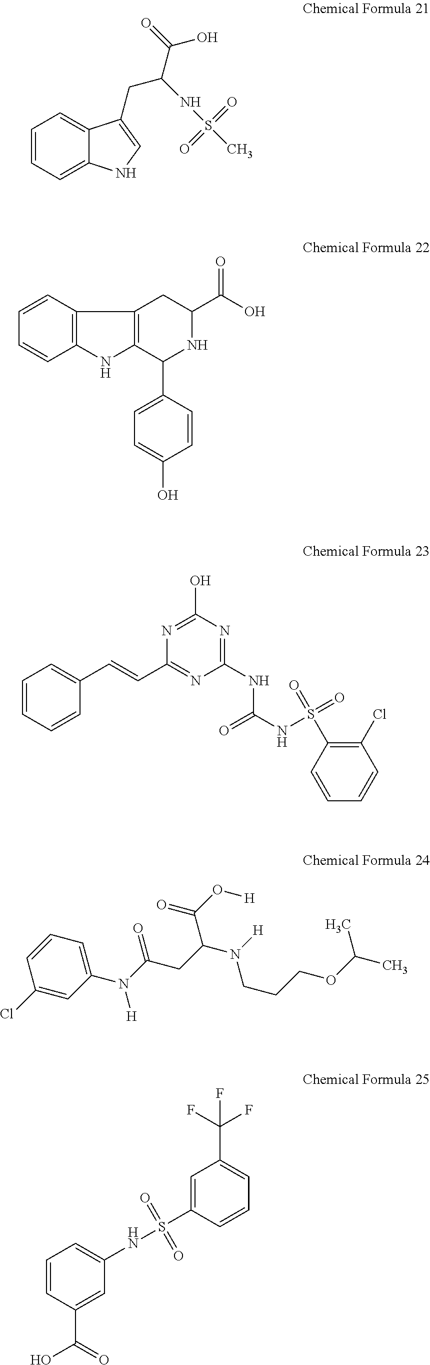 [SMILES: CS(=O)(=O)NC(Cc1c[nH]c2ccccc12)C(=O)O ; O=C(O)C1Cc2c([nH]c3ccccc23)C(c2ccc(O)cc2)N1 ; O=C(Nc1nc(O)nc(/C=C/c2ccccc2)n1)NS(=O)(=O)c1ccccc1Cl ; CC(C)OCCCNC(CC(=O)Nc1cccc(Cl)c1)C(=O)O ; O=C(O)c1cccc(NS(=O)(=O)c2cccc(C(F)(F)F)c2)c1]

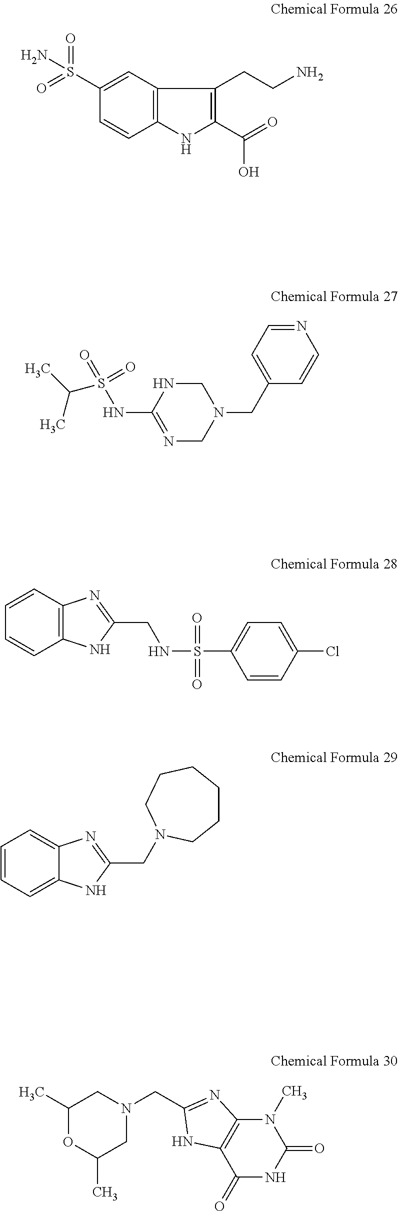 [SMILES: NCCc1c(C(=O)O)[nH]c2ccc(S(N)(=O)=O)cc12 ; CC(C)S(=O)(=O)NC1=NCN(Cc2ccncc2)CN1 ; O=S(=O)(NCc1nc2ccccc2[nH]1)c1ccc(Cl)cc1 ; c1ccc2[nH]c(CN3CCCCCC3)nc2c1 ; CC1CN(Cc2nc3c([nH]2)c(=O)[nH]c(=O)n3C)CC(C)O1]

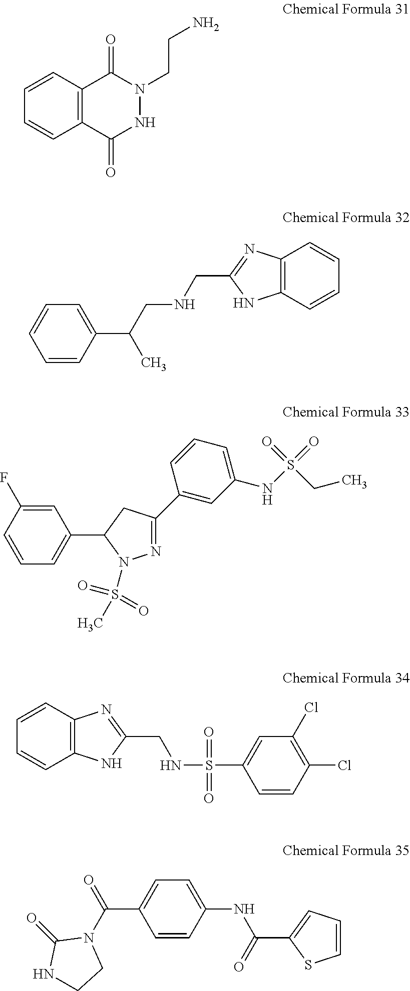 [SMILES: NCCn1[nH]c(=O)c2ccccc2c1=O ; CC(CNCc1nc2ccccc2[nH]1)c1ccccc1 ; CCS(=O)(=O)Nc1cccc(C2=NN(S(C)(=O)=O)C(c3cccc(F)c3)C2)c1 ; O=S(=O)(NCc1nc2ccccc2[nH]1)c1ccc(Cl)c(Cl)c1 ; O=C(Nc1ccc(C(=O)N2CCNC2=O)cc1)c1cccs1]

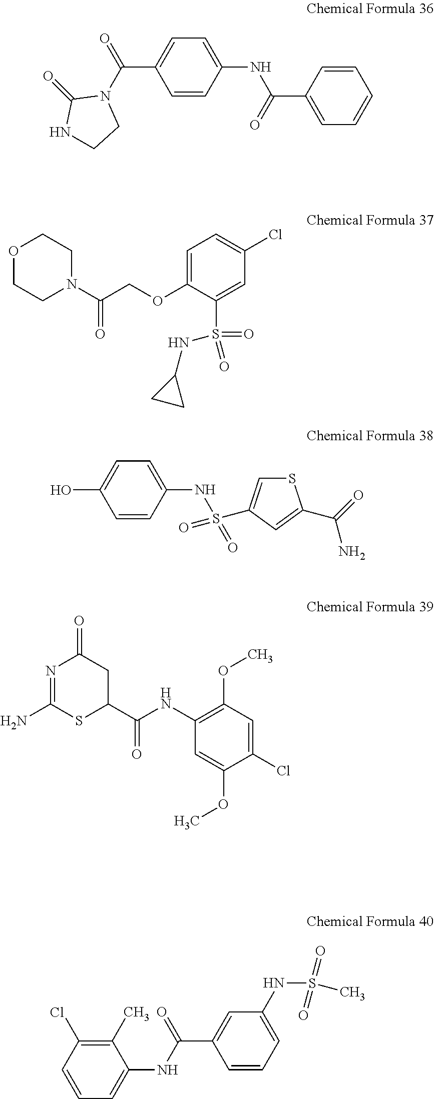 [SMILES: O=C(Nc1ccc(C(=O)N2CCNC2=O)cc1)c1ccccc1 ; O=C(COc1ccc(Cl)cc1S(=O)(=O)NC1CC1)N1CCOCC1 ; NC(=O)c1cc(S(=O)(=O)Nc2ccc(O)cc2)cs1 ; COc1cc(NC(=O)C2CC(=O)N=C(N)S2)c(OC)cc1Cl ; Cc1c(Cl)cccc1NC(=O)c1cccc(NS(C)(=O)=O)c1]

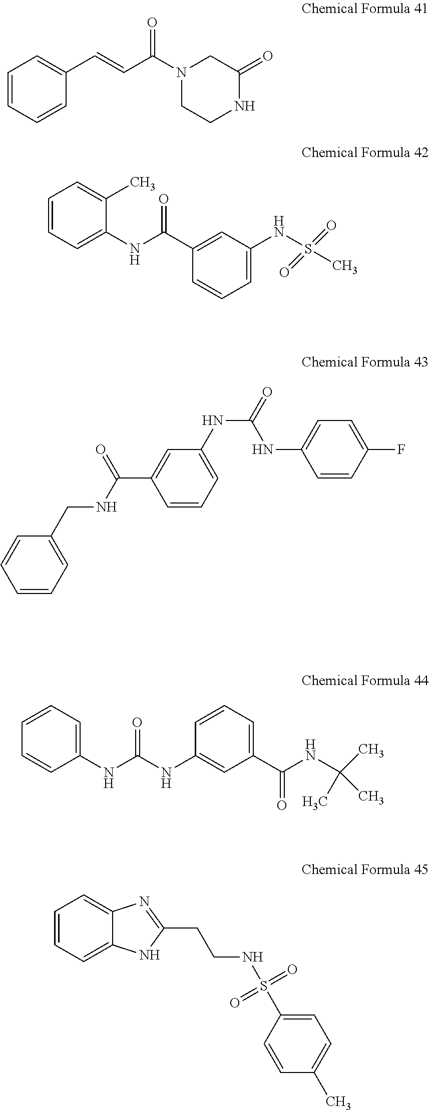 [SMILES: O=C1CN(C(=O)/C=C/c2ccccc2)CCN1 ; Cc1ccccc1NC(=O)c1cccc(NS(C)(=O)=O)c1 ; O=C(Nc1ccc(F)cc1)Nc1cccc(C(=O)NCc2ccccc2)c1 ; CC(C)(C)NC(=O)c1cccc(NC(=O)Nc2ccccc2)c1 ; Cc1ccc(S(=O)(=O)NCCc2nc3ccccc3[nH]2)cc1]

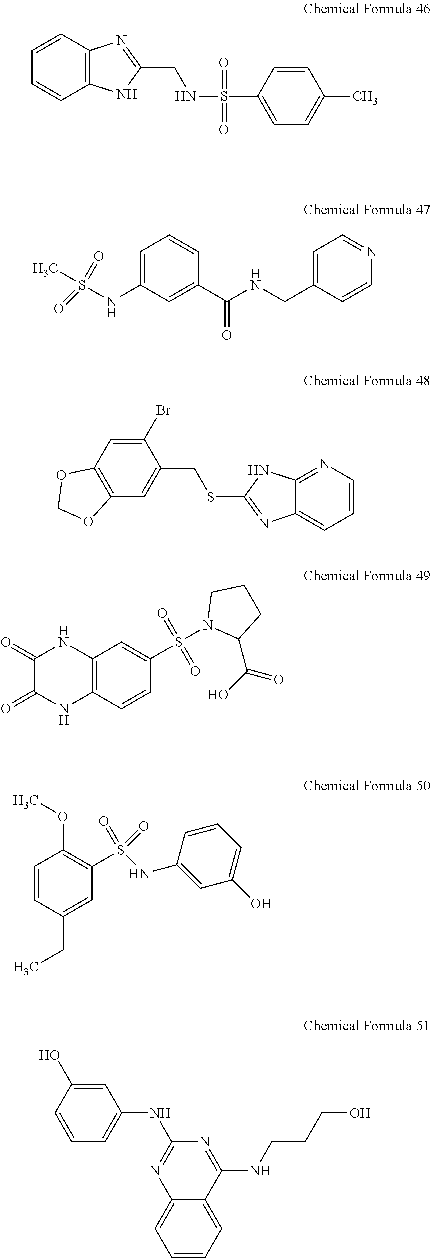

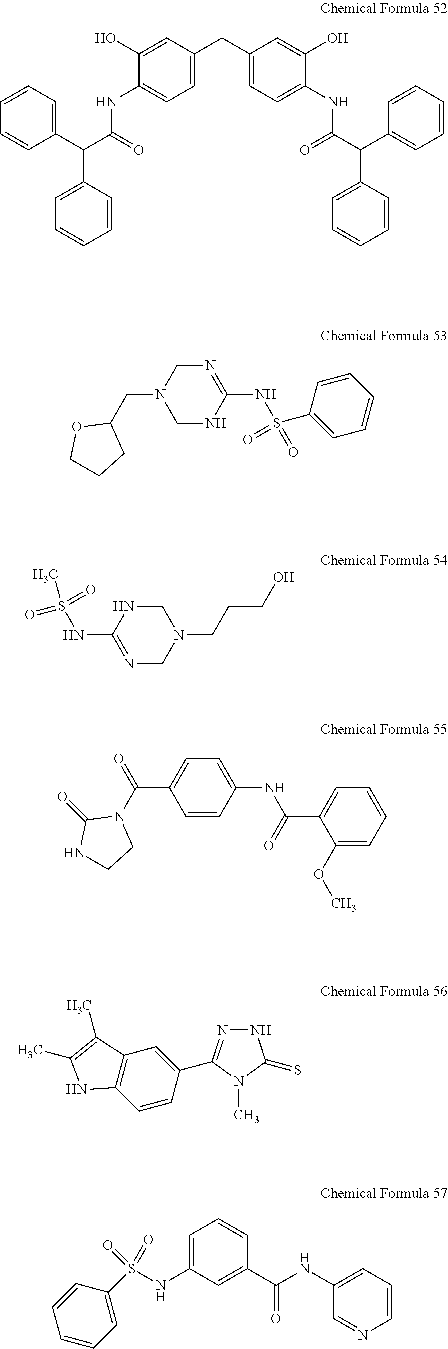 [SMILES: O=C(Nc1ccc(Cc2ccc(NC(=O)C(c3ccccc3)c3ccccc3)c(O)c2)cc1O)C(c1ccccc1)c1ccccc1 ; O=S(=O)(NC1=NCN(CC2CCCO2)CN1)c1ccccc1 ; CS(=O)(=O)NC1=NCN(CCCO)CN1 ; COc1ccccc1C(=O)Nc1ccc(C(=O)N2CCNC2=O)cc1 ; Cc1[nH]c2ccc(-c3n[nH]c(=S)n3C)cc2c1C ; O=C(Nc1cccnc1)c1cccc(NS(=O)(=O)c2ccccc2)c1]

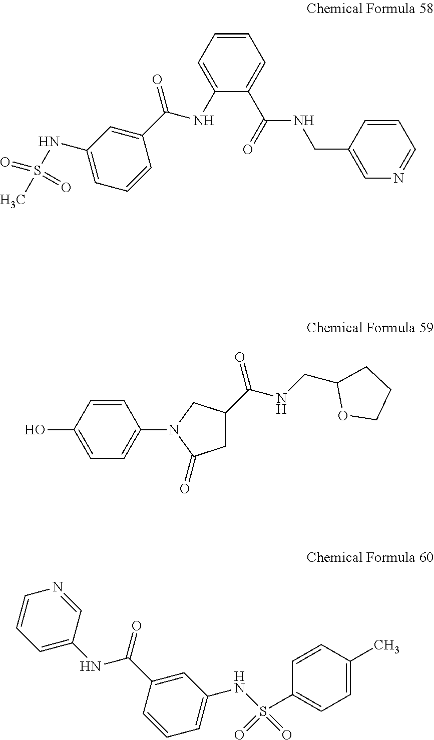 [SMILES: CS(=O)(=O)Nc1cccc(C(=O)Nc2ccccc2C(=O)NCc2cccnc2)c1 ; O=C(NCC1CCCO1)C1CC(=O)N(c2ccc(O)cc2)C1 ; Cc1ccc(S(=O)(=O)Nc2cccc(C(=O)Nc3cccnc3)c2)cc1]

2. A pharmaceutical composition for prevention or treatment of STAT3-related diseases selected from the group consisting of cancer, diabetic retinopathy, diabetes, hemophilic arthrosis, atherosclerosis, keloid, wound granulation, vascular adhesion, autoimmune diseases, restenosis, intestinal adhesions, cat scratch diseases, ulcers, cirrhosis, diabetic nephropathy, malignant neurosis, thrombotic microangiopathy, organ transplant rejection, glomerulopathy, neurodegenerative diseases and inflammatory diseases, comprising:
(a) a pharmaceutically effective amount of the compound according to claim 1, or a pharmaceutically acceptable salt, solvate or hydrate of the compound; and
(b) a pharmaceutically acceptable carrier.

3. The composition according to claim 2, wherein the compound selectively inhibits activity of STAT3 (signal transducer and activator of transcription 3).

4. The composition according to claim 2, wherein the composition inhibits metastasis of cancer cells.

5. The composition according to claim 2, wherein the STAT3-related diseases are cancer selected from the group consisting of gastric cancer, colorectal cancer, lung cancer, human malignant breast cancer, ovarian cancer, liver cancer, bronchial cancer, nasopharyngeal cancer, laryngeal cancer, pancreatic cancer, bladder cancer, colon cancer, cervical cancer, brain cancer, prostate cancer, bone cancer, skin cancer, thyroid cancer, leukemia, lymphoma, adrenal cortical cancer, parathyroid cancer, ureteric cancer, glioma, esophageal cancer, small intestine cancer, glioblastoma, brain tumor and kidney cancer.

6. The composition according to claim 2, wherein the STAT3-related diseases are autoimmune diseases selected from the group consisting of alopecia greata, ankylosing spondylitis, antiphospholipid syndrome, autoimmune Addison disease, adrenal autoimmune disease, autoimmune hemolytic anemia, autoimmune hepatitis, autoimmune ovaryitis and orchitis, autoimmune thrombocytopenia, Behcet's disease, vesicular ichthyosis, cardiomyopathy, celiac sprue-dermatitis, chronic fatigue immunodeficiency syndrome, chronic inflammatory demyelinating multiple neuropathy, Chur-strauss syndrome, reflexive Yucheonpochang, CREST syndrome, cold agglutinin disease, Crohn's disease, discous lupus, abdominal complex cold globulinemia, fibromyalgia-fibromyositis, glomerulonephritis, Graves' disease, Schwain-Barre syndrome, Hashimoto's thyroiditis, idiopathic pulmonary fibrosis, idiopathic thrombocytopenic purpura, IgA neuritis, combustive arthritis, lichen planus, lupus erythematosus, Menirre's disease, mixed connective tissue disease, multiple sclerosis, type I or immune-mediated diabetes, myasthenia gravis, vulgar astrocyst, malignant anemia, crystalline polyarteritis, Badal-chondritis, autoimmune polyline syndrome, rheumatoid multiple myalgia, multiple myositis and dermatomyositis, primary agamma globulinemia, primary glycemic cirrhosis, psoriasis, psoriatic arthritis, Raynaud's phenomenon, Reiter's syndrome, rheumatoid arthritis, sarcoidosis, scleroderma, ankylosing human syndrome, systemic lupus erythematosus, lupus erythematosus, Takayasu's arteritis, transient arteritis, giant cell arteritis, ulcerative colitis, uveitis, vitiligo, and Wegener's granulomatosis.

7. The composition according to claim 2, wherein the STAT3-related diseases are inflammatory diseases selected from the group consisting of asthma, encephalitis, inflammatory enteritis, rheumatoid arthritis, chronic obstructive pulmonary disease, allergic, atopic dermatitis, psoriasis, pulmonary thrombosis, pefiosis, undifferentiated spinal joint disease, Crohn's disease, pancreatitis, dermatitis, undifferentiated arthrosis, arthritis, glomerulonephritis, bronchitis, inflammatory osteolysis, and chronic inflammation caused by viral or bacterial infection.

8. A food composition for prevention or improvement of STAT3-related diseases selected from the group consisting of cancer, diabetic retinopathy, diabetes, hemophilic arthrosis, atherosclerosis, keloid, wound granulation, vascular adhesion, autoimmune diseases, restenosis, intestinal adhesions, cat scratch diseases, ulcers, cirrhosis, diabetic nephropathy, malignant neurosis, thrombotic microangiopathy, organ transplant rejection, glomerulopathy, neurodegenerative diseases and inflammatory diseases, comprising the compound according to claim 1 or a salt of the compound as an active ingredient.

9. A method of preventing or treating STAT3-related diseases selected from the group consisting of cancer, diabetic retinopathy, diabetes, hemophilic arthrosis, atherosclerosis, keloid, wound granulation, vascular adhesion, autoimmune diseases, restenosis, intestinal adhesions, cat scratch diseases, ulcers, cirrhosis, diabetic nephropathy, malignant neurosis, thrombotic microangiopathy, organ transplant rejection, glomerulopathy, neurodegenerative diseases and inflammatory diseases, the method comprising administering (a) a pharmaceutically effective amount of the compound according to claim 1, or a pharmaceutically acceptable salt, solvate or hydrate of the compound; and (b) a pharmaceutically acceptable carrier to an individual.

10. Use of the compound according to claim 1, or a pharmaceutically acceptable salt, solvate or hydrate of the compound to be used in a pharmaceutical composition for prevention or treatment of STAT3-related diseases selected from the group consisting of cancer, diabetic retinopathy, diabetes, hemophilic arthrosis, atherosclerosis, keloid, wound granulation, vascular adhesion, autoimmune diseases, restenosis, intestinal adhesions, cat scratch diseases, ulcers, cirrhosis, diabetic nephropathy, malignant neurosis, thrombotic microangiopathy, organ transplant rejection, glomerulopathy, neurodegenerative diseases and inflammatory diseases.

11. Use of the compound according to claim 1, or a pharmaceutically acceptable salt, solvate or hydrate of the compound to be used in a food composition for prevention or improvement of STAT3-related diseases selected from the group consisting of cancer, diabetic retinopathy, diabetes, hemophilic arthrosis, atherosclerosis, keloid, wound granulation, vascular adhesion, autoimmune diseases, restenosis, intestinal adhesions, cat scratch diseases, ulcers, cirrhosis, diabetic nephropathy, malignant neurosis, thrombotic microangiopathy, organ transplant rejection, glomerulopathy, neurodegenerative diseases and inflammatory diseases.
